SEQUENCE LISTING (55 sequences)

SEQ ID NO: 1 - DNA, 10001 bp, Mus musculus
tgactatcagcctgtatggtcattaagttctgtctaccctgggaaagcctctgcccaaacctccctattcacaggtcctagaacgtagaggtggggagcaggacggtgccgccaggccgtgtgcgatcgcgagctctggatctcaatgcgccccggggcgctgtttcccacgactccagcagcttttctaaaaatccaggcagcctccagtttacgggatcaacccgagactcgcttccctttgaaaattctagagtataaagtaaacgtacgagcaaagtatgtgtcttaacacttaatggatgacatagagaccaaaaaagccatgtccgtgggcccagtaggccgcaataaggggcgaccaggaaactgcagcacagccccccgcagccgccctgctcccacaccagtcattccagcaccgtggtgaaggcgcttgggggcggggcggggcgcgcctgcgcagcgaggctctgcagcagaaactttgcctagaccggctggaaccggttagaaccggtcgaacccggccggctgccagccctcgattcagcagctcacaaagggaggcggcgactcacgacccgcgtatccttgcgcctctccccaccccctttgtcctcgcgacgggttccgcggtcctccccgccctccctggcgcggcccccgctttctgcgcccagtgacgctttctccatggtcctgggagaaagagaaaaacatcctttcccctccgtcgtagttttaggaagcgatgagatagacctggggaccttgccgccacgggccgggctctgacggttattagcgcagtgcgggtggtgctcggcatggccgccaaggtcgccgtgccctcacctgcagcgaccatggccttgctgggctgagaccgcagcctaacatggcggacgtaggcaagcaccaaagcgctcgtgtacccgggctcggaaaagtggccccgagagcagccggaggctgcaggtcgtccctacaggagcattcccagtataaaccagtacaaagtgtcaccacctcagaagccactcgcagggccggtcactttccgagagacctccatcttgtttcgcatgaaatggcagccgctcggggagttacaaaatgggaagtggaagctgaagctgtgggaaagcctgttttaacacttgcaacatacgctataccctctgtcctcccaggaaaacgcaaaaggtgttgaaacatctgaaaaacttggggctcccatttttaatagctattagttcatgttttttctccttgtgaccagaaattttaaacctatttgtacctatttagctggtacaagctaaacatttctctgtattagcaaggtccaagaggcccacacgacgtcaagaaaaatctagaaacttggaagtcaggatctatttttaactctctgaggaactatttttcttccttcaccaaggtggtggagggttactaggttccggtggagtgacgtgtccctttgcaataaataccggcgctccgggctctgcgtcaggcattcaggcagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttcgactggcgcatgtacgtttgaaggcatgagttggaaacagggaagatggaagtgttaggctagccgggcgatggtggcgcacgcctttaatcctagcacttgggaggcagaggcaggcggatttctgagttcgaggccagcctggtctacagagtgagttccaggacagccagggctacacagagaaaccctgtcttgaaaaaacaaaaaggttaggctagtatttggagaaagaagattagaaaatggaagtgaaagacgaagaagacatacaggaaggtgaagaaaaagctgttagagaagataggaaaatagaagacaaagcatctttagaagacagaaaaggtacttaaaggcacaggtagtaggaagccgaagaatagaagatagaaagaagcaagatagaaaaacaaaatggaagttaagacaactttggatgccagcattcaagataggcaaagaagataagattgaggccaaaaggttggataagatataaagtcagaaggaaattatctttaaagccataagttcaaatttctgatggagcgagcagtttagaagagtctttagacagccacatacaagattgaagctagcaatcaaagctactaggactgaagtaaaaagttaaggcagaatgcctttgaagagttagaagaatattaaaagccttaacttgtagcttaattttgcttgatgacaaaaggacttttgataacagtttcaagattgtcagcattttgcattggacttgagctgaggtgcttttaaaatcctaacgactagcattggcagctgacccaggtctacacagaagtgcattcagtgaactaggaagacaggagcggcagacaggagtcccgaagccagtttggtgaagctaggaaggactgaggagccagcagcagcagtgcatggtgaagatagcccaggaaagagtgcggttcggtggaggaagctaggaagaaggagccatacggatgtggtggtgaagctgggaaagggttccaggatggtggagcgagagcgagttggtgatgaagctagctggcggcttggcttgtcaactgcgcggaggaggcgagcaggcattgtggagaggatagatagcggctcctagaccagcatgccagtgtgcaagaaaggctgcagggagagcatgcggtgcggtaacattccttgaggtcggcaacatggtggtggttttctgtaacttggatggtaacttgtttactttgtcttaatagttatgggggagttgtaggcttctgtgtaaagagatatatctggggctgtatgtaggcctttgcgggtgttgtaggtttttctttttcagggttatgtcctcttgcatcttgtcagaagcttttgagggctgactgccaaggcccagaaagaagaatggtagatggcaagttgtctttaaccgctcagaggggaatgaatggtagagccagcacaacctcccagttttgtaagacgttgtagtttgaacagatgacctaccacaagcctcactcctgtgtaggggaggtaattgggcaaagtgcttttgggggaatgggggcaaaatatattttgagttcttttccccttaggtctgtctagaatcctaaaggcagatgactcaagggaaccagaaaaaaggaaatccactctcaggataagcagagctcgccaggtttacagtttgtaggaagtagaggatggatgctagctttcacactgagtgtggaggagctggccatggcggaattgctggtagtttactctttccccctcccttaatgagatttgtaaaatcctaaacacttttacttgaaatatttgggagtggtcttaacagggaggagtgggtgggggaaacgttttttttctaagattttccacagatgctatagttgtgttgacacactgggttagagaaggcgtgtactgctatgctgttggcacgacaccttcagggactggagctgccttttgtccttggaagagttttcccagttgccgctgaagtcagcacagtgcggctttggttcacagtcacctcaggagaacctcaggagcttggctaggccagaggttgaagttaagttttacagcaccgtgatttaaaatatttcattaaaggggaggggtaaaacttagttggctgtggccttgtgtttgggtgggtgggggtgttaggtaattgtttagtttatgatttcagataatcataccagagaacttaaatatttggaaaaacaggaaatctcagctttcaagttggcaagtaactcccaatccagtttttgcttcttttttcctttttctttttttgaggcgggcagctaaggaaggttggttcctctgccggtccctcgaaagcgtagggcttgggggttggtctggtccactgggatgatgtgatgctacagtggggactcttctgaagctgttggatgaatatagattgtagtgtgtggttctcttttgaaatttttttcaggtgacttaatgtatcttaataactactataggaacaaaggaagtggctttaatgaccctgaaggaatttcttctggtgatagcttttatattatcaagtaagagatactatctcagttttgtataagcaagtctttttcctagtgtaggagaaatgattttccttgtgactaaacaagatgtaaaggtatgctttttttcttcttgtgcattgtatacttgtgtttatttgtaacttataatttaagaattatgataattcagcctgaatgtcttttagagggtgggcttttgttgatgagggaggggaaacctttttttttctgtagacctttttcagataacaccatctgagtcataaccagcctggcagtgtgatgacgtagatgcagagggagcagctccttggtgaatgagtgataagtaaaggcagaaaaaataatgtcatgtctccatggggaatgagcatgagccagagattgttcctactgatgaaaagctgcatatgcaaaaatttaagcaaatgaaagcaaccagtataaagttatggcaatacctttaaaagttatggcttatctaccaagctttatccacaaaagtaaagaattgatgaaaaacagtgaagatcaaatgttcatctcaaaactgcttttacaaaagcagaatagaaatgaagtgaaaatgctgcattaagcctggagtaaaaagaagctgagcttgttgagatgagtgggatcgagcggctgcgaggcggtgcagtgtgccaatgtttcgtttgcctcagacaggtttctcttcataagcagaagagttgcttcattccatctcggagcaggaaacagcagactgctgttgacagataagtgtaacttggatctgcagtattgcatgttagggatagataagtgccttttttctctttttccaaaaagacctgtagagctgttgaatgtttgcagctggcccctcttaggcagttcagaattttgagtagttttcccatccagcctcttaaaaattcctaagccttgcaccgatgggctttcatgatgggatagctaataggcttttgcatcgtaaacttcaacacaaaagcctacatgattaatgcctactttaattacattgcttacaagattaaggaatctttatcttgaagaccccatgaaagggatcattatgtgctgaaaattagatgttcatattgctaaaatttaaatgtgctccaatgtacttgtgcttaaaatcattaaattatacaaattaataaaatacttcactagagaatgtatgtatttagaaggctgtctccttatttaaataaagtcttgtttgttgtctgtagttagtgtgggcaattttggggggatgttcttctctaatcttttcagaaacttgacttcgaacacttaagtggaccagatcaggatttgagccagaagaccgaaattaactttaaggcaggaaagacaaattttattctccatgcagtgatgagcatttaataattgcaggcctggcatagaggccgtctaactaaggactaagtaccttaggcaggtgggagatgatggtcagagtaaaaggtaactacatattttgtttccagaaagtcaggggtctaatttgaccatggctaaacatctagggtaagacacttttcccccacatttccaaatatgcatgttgagtttaaatgcttacgatcatctcatccactttagccttttgtcacctcacttgagccacgagtggggtcaggcatgtgggtttaaagagttttcctttgcagagcctcatttcatccttcatggagctgctcaggactttgcatataagcgcttgcctctgtcttctgttctgctagtgagtgtgtgatgtgagaccttgcagtgagtttgtttttcctggaatgtggagggagggggggatggggcttacttgttctagctttttttttacagaccacacagaatgcaggtgtcttgacttcaggtcatgtctgttctttggcaagtaatatgtgcagtactgttccaatctgctgctattagaatgcattgtgacgcgactggagtatgattaaagaaagttgtgtttccccaagtgtttggagtagtggttgttggaggaaaagccatgagtaacaggctgagtgttgaggaaatggctctctgcagctttaagtaacccgtgtttgtgattggagccgagtccctttgctgtgctgccttaggtaaatgtttttgttcatttctggtgaggggggttgggagcactgaagcctttagtctcttccagattcaacttaaaatctgacaagaaataaatcagacaagcaacattcttgaagaaattttaactggcaagtggaaatgttttgaacagttccgtggtctttagtgcattatctttgtgtaggtgttctctctcccctcccttggtcttaattcttacatgcaggaacattgacaacagcagacatctatctattcaaggggccagagaatccagacccagtaaggaaaaatagcccatttactttaaatcgataagtgaagcagacatgccattttcagtgtggggattgggaagccctagttctttcagatgtacttcagactgtagaaggagcttccagttgaattgaaattcaccagtggacaaaatgaggacaacaggtgaacgagccttttcttgtttaagattagctactggtaatctagtgttgaatcctctccagcttcatgctggagcagctagcatgtgatgtaatgttggccttggggtggaggggtgaggtgggcgctaagcctttttttaagatttttcaggtacccctcactaaaggcactgaaggcttaatgtaggacagcggagccttcctgtgtggcaagaatcaagcaagcagtattgtatcgagaccaaagtggtatcatggtcggttttgattagcagtggggactaccctaccgtaacaccttgttggaattgaagcatccaaagaaaatacttgagaggccctgggcttgttttaacatctggaaaaaaggctgtttttatagcagcggttaccagcccaaacctcaagttgtgcttgcaggggagggaaaagggggaaagcgggcaaccagtttccccagcttttccagaatcctgttacaaggtctccccacaagtgatttctctgccacatcgccaccatgggcctttggcctaatcacagacccttcacccctcaccttgatgcagccagtagctggatccttgaggtcacgttgcatatcggtttcaaggtaaccatggtgccaaggtcctgtgggttgcaccagaaaaggccatcaattttccccttgcctgtaatttaacattaaaaccatagctaagatgttttatacatagcacctatgcagagtaaacaaaccagtatgggtatagtatgtttgataccagtgctgggtgggaatgtaggaagtcggatgaaaagcaagcctttgtaggaagttgttggggtgggattgcaaaaattctctgctaagactttttcaggtggacataacagacttggccaagctagcatcttagtggaagcagattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgcttgactcttatatcatgaggccattacatttttcttggagggttctaaaggctctgggtatggtagctgatatcactggaacactccccagcctcagtgttgaactcttgataattaactgcattgtctttcaggttatgcccaattcgtcttattacctctgagtcgacacacctcctactatttattgaatactttgattttatgaaataaaaactaaatatctctcattgtgtgcttctttgtgcataaaacacaggcttattttaagcctaaagagaccaaatgtctgatctacctcagcttctccgattagtgaggccttccctgtttccttgggctgcatggctctttcatgcagatggctctaaagttgggcttgggtcctaggtggccactcttgcacctcaggaacacaaggcctttccctgctgttcaggctctcctccctgagaaaacattctggattgtctatgaggaagttgggaaaagatggtgtcgaaaagaggtggtgtgcattgctcctctgttcctaacactggatggaagactagttttcatgtagtttagggaaatagttatacatggtctaaaggcccaaaaacattcccagagtgtatgcaatactgtgtgtaagtgtgcactgcgtgtgtttggaggtcagaacttctctgaggttctagagatgaagcaagtcctcagccatggcccaagaatgggaaggaactgggtcctgctgtaccacttcccattccttaaggaacagtttggcccggtgtggtgcaagcatggtcggtcactgaaaaaagaaaacccacttaggtttcacaggcttgaagagctgcatgtcatccagcaaattactggctgctgtaaggacaggcccctaggtcccagtcccaggtgcccttcctgccactcaatcaagccttacaccctgggcaaaaacatcctgcgttgaaggttcagctcccagggctggaaacttgtgctggcatctaccccagttcaaaggggctcagcacattgacaactaaaactaagccctcaggtgagcaaaatggtctccttaaggcaatcatggtcattggtgttcctgcagtaaaggacagcatcacagctgatgtctgtgtactggctagttttgtatcaacttgacacagctggaattatcacagagaaagcttcagttggggaagtgcctccaagagatcctccacgagatcctgctctaaggcattttctcaattagtgatcaagggggaaagaccccttgtgtgtgggaccatctctgggctggtagtcttggttcagttctataagagagcaggctgagcaagccaggggaagcaagccagtaaagaacatccctccatggcctctgcatcagctcctgcttcctgacctgcttgagttccagtcctgacttccttggtgatgaacagcagtatggaagtgtaagccgaataaaccctgtcctccccaacttgcttcttggtcatgtttgtgcaggaatagaaaccctgactaagacagtctgagacctgacagatctgtgctaaagtctggtaccaactgagctagaccctgccacacacctcagtaatggcccattctgaattcacccagagctgaggctttgccgaggtgaggcacaaagacttcactggagagcaggagatatgaacagaggttggggctcacacttcctgattgggggccagga

SEQ ID NO: 2 - DNA, 17 bp, Artificial sequence
  Primer
aagaggcggcggaaggt

SEQ ID NO: 3 - DNA, 20 bp, Artificial sequence
  Primer
cgggcgaggcgtatttatag

SEQ ID NO: 4 - DNA, 24 bp, Artificial sequence
  Probe
tccggtgatgcgagttgttctccg

SEQ ID NO: 5 - DNA, 23 bp, Artificial sequence
  Primer
tgggttagagaaggcgtgtactg

SEQ ID NO: 6 - DNA, 18 bp, Artificial sequence
  Primer
tcagcggcaactgggaaa

SEQ ID NO: 7 - DNA, 25 bp, Artificial sequence
  Probe
cgttggcacgacaccttcagggact

SEQ ID NO: 8 - DNA, 19 bp, Artificial sequence
  Synthetic oligonucleotide
tcagcattctaatagcagc

SEQ ID NO: 9 - DNA, 16 bp, Artificial sequence
  Synthetic oligonucleotide
gcattctaatagcagc

SEQ ID NO: 10 - protein, 8 aa, Artificial sequence
  Synthetic peptide
  MISC_FEATURE (5)..(5): Xaa is an amino acid selected from Ile and Val
DRVYXHPF

SEQ ID NO: 11 - protein, 8 aa, Artificial sequence
  Synthetic peptide
  MISC_FEATURE (1)..(1): Xaa is an amino acid selected from Asp, Sar, Ala, and NH2
  MISC_FEATURE (2)..(2): Xaa is an amino acid selected from Arg and Gln
  MISC_FEATURE (3)..(3): Xaa is an amino acid selected from Val and Ala
  MISC_FEATURE (4)..(4): Xaa is an amino acid selected from Tyr, Ala, Ile, Gly, Cha
  MISC_FEATURE (5)..(5): Xaa is an amino acid selected from Ile and Val
  MISC_FEATURE (6)..(6): Xaa is an amino acid selected from His and Ala
  MISC_FEATURE (7)..(7): Xaa is an amino acid selected from Pro and Ala
  MISC_FEATURE (8)..(8): Xaa is an amino acid selected from Phe, Ala, Ile, Gly, Cha, and Dip
XXXXXXXX

SEQ ID NO: 12 - protein, 8 aa, Artificial sequence
  Synthetic peptide
DRVYIHPF

SEQ ID NO: 13 - protein, 8 aa, Artificial sequence
  Synthetic peptide
DRVYVHPF

SEQ ID NO: 14 - protein, 9 aa, Artificial sequence
  Synthetic peptide
  MISC_FEATURE (1)..(1): Xaa is N6-(2-azidoacetyl)-lysine
XDRVYIHPF

SEQ ID NO: 15 - protein, 18 aa, Artificial sequence
  Synthetic peptide
  MISC_FEATURE (1)..(1): Xaa is N6-(2-azidoacetyl)-lysine
XPPPAGSSPGDRVYIHPF

SEQ ID NO: 16 - protein, 9 aa, Artificial sequence
  Synthetic peptide
  MISC_FEATURE (1)..(1): Xaa is an amino acid selected from lysine, D-lysine, L-lysine, and N6-(2-azidoacetyl)-lysine
XDRVYIHPF

SEQ ID NO: 17 - protein, 18 aa, Artificial sequence
  Synthetic peptide
  MISC_FEATURE (1)..(1): Xaa is an amino acid selected from lysine, D-lysine, L-lysine, and N6-(2-azidoacetyl)-lysine
XPPPAGSSPGDRVYIHPF

SEQ ID NO: 18-19 - skipped (2 entries)

SEQ ID NO: 20 - protein, 9 aa, Artificial sequence
  Synthetic peptide
PPPAGSSPG

SEQ ID NO: 21 - protein, 9 aa, Artificial sequence
  Synthetic peptide
  misc_feature (1)..(1): Xaa can be any amino acid
XPPAGSSPG

SEQ ID NO: 22 - protein, 9 aa, Artificial sequence
  Synthetic peptide
  misc_feature (2)..(2): Xaa can be any amino acid
PXPAGSSPG

SEQ ID NO: 23 - protein, 9 aa, Artificial sequence
  Synthetic peptide
  misc_feature (3)..(3): Xaa can be any amino acid
PPXAGSSPG

SEQ ID NO: 24 - protein, 9 aa, Artificial sequence
  Synthetic peptide
  misc_feature (4)..(4): Xaa can be any amino acid
PPPXGSSPG

SEQ ID NO: 25 - protein, 9 aa, Artificial sequence
  Synthetic peptide
  misc_feature (5)..(5): Xaa can be any amino acid
PPPAXSSPG

SEQ ID NO: 26 - protein, 9 aa, Artificial sequence
  Synthetic peptide
  misc_feature (6)..(6): Xaa can be any amino acid
PPPAGXSPG

SEQ ID NO: 27 - protein, 9 aa, Artificial sequence
  Synthetic peptide
  misc_feature (7)..(7): Xaa can be any amino acid
PPPAGSXPG

SEQ ID NO: 28 - protein, 9 aa, Artificial sequence
  Synthetic peptide
  misc_feature (8)..(8): Xaa can be any amino acid
PPPAGSSXG

SEQ ID NO: 29 - protein, 9 aa, Artificial sequence
  Synthetic peptide
  misc_feature (9)..(9): Xaa can be any amino acid
PPPAGSSPX

SEQ ID NO: 30 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
XPPPAGSSPG

SEQ ID NO: 31 - protein, 10 aa, Artificial sequence
  Synthetic peptide
  misc_feature (1)..(1): Xaa can be any amino acid
XPPPAGSSPG

SEQ ID NO: 32 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (2)..(2): Xaa can be any amino acid
XXPPAGSSPG

SEQ ID NO: 33 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (3)..(3): Xaa can be any amino acid
XPXPAGSSPG

SEQ ID NO: 34 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (4)..(4): Xaa can be any amino acid
XPPXAGSSPG

SEQ ID NO: 35 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (5)..(5): Xaa can be any amino acid
XPPPXGSSPG

SEQ ID NO: 36 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (6)..(6): Xaa can be any amino acid
XPPPAXSSPG

SEQ ID NO: 37 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (7)..(7): Xaa can be any amino acid
XPPPAGXSPG

SEQ ID NO: 38 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (8)..(8): Xaa can be any amino acid
XPPPAGSXPG

SEQ ID NO: 39 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (9)..(9): Xaa can be any amino acid
XPPPAGSSXG

SEQ ID NO: 40 - protein, 10 aa, Artificial sequence
  Synthetic peptide
  MISC_FEATURE (1)..(1): Xaa is an amino acid selected from lysine, D-lysine, L-lysine, and N6-(2-azidoacetyl)-lysine
  misc_feature (10)..(10): Xaa can be any amino acid
XPPPAGSSPX

SEQ ID NO: 41 - protein, 10 aa, Artificial sequence
  Synthetic peptide
CPPPAGSSPG

SEQ ID NO: 42 - protein, 10 aa, Artificial sequence
  Synthetic peptide
  misc_feature (2)..(2): Xaa can be any amino acid
CXPPAGSSPG

SEQ ID NO: 43 - protein, 10 aa, Artificial sequence
  Synthetic peptide
  misc_feature (3)..(3): Xaa can be any amino acid
CPXPAGSSPG

SEQ ID NO: 44 - protein, 10 aa, Artificial sequence
  Synthetic peptide
  misc_feature (4)..(4): Xaa can be any amino acid
CPPXAGSSPG

SEQ ID NO: 45 - protein, 10 aa, Artificial sequence
  Synthetic peptide
  misc_feature (5)..(5): Xaa can be any amino acid
CPPPXGSSPG

SEQ ID NO: 46 - protein, 10 aa, Artificial sequence
  Synthetic peptide
  misc_feature (6)..(6): Xaa can be any amino acid
CPPPAXSSPG

SEQ ID NO: 47 - protein, 10 aa, Artificial sequence
  Synthetic peptide
  misc_feature (7)..(7): Xaa can be any amino acid
CPPPAGXSPG

SEQ ID NO: 48 - protein, 10 aa, Artificial sequence
  Synthetic peptide
  misc_feature (8)..(8): Xaa can be any amino acid
CPPPAGSXPG

SEQ ID NO: 49 - protein, 10 aa, Artificial sequence
  Synthetic peptide
  misc_feature (9)..(9): Xaa can be any amino acid
CPPPAGSSXG

SEQ ID NO: 50 - protein, 10 aa, Artificial sequence
  Synthetic peptide
  misc_feature (10)..(10): Xaa can be any amino acid
CPPPAGSSPX

SEQ ID NO: 51 - protein, 15 aa, Artificial sequence
  Synthetic peptide
CAGSIKPPPAGSSPG

SEQ ID NO: 52 - protein, 15 aa, Artificial sequence
  Synthetic peptide
KAGSIKPPPAGSSPG

SEQ ID NO: 53 - protein, 10 aa, Artificial sequence
  Synthetic peptide
  MISC_FEATURE (1)..(1): Xaa is selected from lysine, D-lysine, L-lysine, and N6-(2-azidoacetyl)-lysine, and cysteine
XPAPSGPSPG

SEQ ID NO: 54 - protein, 15 aa, Artificial sequence
  Synthetic peptide
  MISC_FEATURE (1)..(1): Xaa is selected from lysine, D-lysine, L-lysine, and N6-(2-azidoacetyl)-lysine, and cysteine
XAGSIKPPPAGSSPG

SEQ ID NO: 55 - protein, 10 aa, Artificial sequence
  Synthetic peptide
  MISC_FEATURE (1)..(1): Xaa is selected from lysine, D-lysine, L-lysine, and N6-(2-azidoacetyl)-lysine, and cysteine
XAGMSGASAG

(19) United States
(12) Patent Application Publication
(10) Pub. No.: US 2022/0243210 A1
(43) Pub. Date: Aug. 4, 2022

(54) ANGIOTENSIN II TYPE 1 RECEPTOR TARGETED OLIGONUCLEOTIDES AND USES THEREOF

(71) Applicant: Ionis Pharmaceuticals, Inc., Carlsbad, CA (US)

(72) Inventors: Adam Mullick, Carlsbad, CA (US); Yichun Kuo, San Diego, CA (US); Ting Yuan Yeh, San Marcos, CA (US); Thazha P. Prakash, Carlsbad, CA (US)

(73) Assignee: Ionis Pharmaceuticals, Inc., Carlsbad, CA (US)

(21) Appl. No.: 17/611,780

(22) PCT Filed: May 18, 2020
(86) PCT No.: PCT/US20/33476

§ 371 Date: Nov. 16, 2021

Related U.S. Application Data
(60) Provisional application No. 62/849,812, filed on May 17, 2019.

(51) Int. Cl. C12N 15/113; C12N 15/11

(52) U.S. Cl. CPC C12N 15/1138; C12N 15/111; C12N 2310/11; C12N 2320/32; C12N 2310/3231; C12N 2310/3341; C12N 2310/341; C12N 2310/315

(57) ABSTRACT

The present disclosure provides compounds and methods for targeting cells expressing AGTR1. In some instances, the compound includes an oligonucleotide and an AGTR1 binding conjugate moiety, and optionally a conjugate linker.

### SEQUENCE LISTING

The present application is being filed along with a Sequence Listing in electronic format. The Sequence Listing is provided as a file entitled CORE0157WOSEQ_ST25.txt created on May 18, 2020 which is 32 KB in size. The information in the electronic format of the sequence listing is incorporated herein by reference in its entirety.

### FIELD

The present embodiments provide compounds and methods for targeting cells expressing angiotensin II type I receptor (AGTR1).

### BACKGROUND

Angiotensin II (Ang II) peptide hormone (Asp-Arg-Val-Tyr-Ile/Val-His-Pro-Phe (SEQ ID NO: 10)) and analogs thereof can function as a potent vasopressor hormone and a primary regulator of aldosterone secretion, thereby functioning as a key modulator of blood pressure, blood volume, and ultimately cardiovascular system health and function. Cardiovascular effects of Ang II are primarily mediated by angiotensin II type I receptor (AGTR1). AGTR1 is expressed in a variety of tissues including heart and kidney, and highly expressed in liver, adrenal gland and adipose tissue. Ang II interaction with AGTR1 results in rapid AGTR1 internalization and AGTR1-mediated endocytosis of intact Ang II is the principal route of Ang II plasma clearance.

### SUMMARY

Embodiments provided herein are directed to compounds and methods for modulating the expression of a nucleic acid target in cells expressing AGTR1. In certain embodiments, a compound comprises an oligonucleotide and an AGTR1 binding conjugate moiety. In certain embodiments, a compound comprises an oligonucleotide, conjugate linker, and an AGTR1 binding cell-targeting moiety. In certain embodiments, contacting a cell expressing AGTR1 with a compound provided herein modulates expression of a nucleic acid target in the cell. In certain embodiments, a compound comprising a AGTR1 binding cell-targeting moiety selectively or preferentially targets a cell expressing AGTR1 compared to a cell not expressing AGTR1. In certain embodiments, a compound comprising a AGTR1 binding cell-targeting moiety selectively or preferentially targets a cell expressing AGTR1 compared to a compound not comprising a AGTR1 binding cell-targeting moiety.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

Oligonucleotides described by Compound No. indicate a combination of nucleobase sequence, chemical modification, and motif.

It is understood that throughout the specification, the first letter in a peptide sequence is the first amino acid of the peptide at the N-terminus and the last letter in a peptide sequence is the last amino acid of the peptide at the C-terminus unless indicated otherwise.

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil). A β-D-2′-deoxyribosyl sugar moiety or 2′-β-D-deoxyribosyl sugar moiety in the context of an oligonucleotide is an unsubstituted, unmodified 2′-deoxyfuranosyl and is found in naturally occurring deoxyribonucleic acids (DNA).

“2′-O-methoxyethyl” (also 2′-MOE and 2′-O(CH2)2-OCH3) refers to an O-methoxy-ethyl modification at the 2′ position of a furanosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2-substituent group other than H or OH.

“5-methylcytosine” means a cytosine with a methyl group attached to the 5 position.

“About” means within 10% of a value. For example, if it is stated, “the compounds affected about 70% inhibition of a target nucleic acid”, it is implied that target nucleic acid levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Aminoisobutyric acid” or “Aib” means 2-aminoisobutyric acid having the formula:

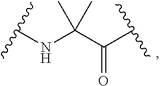 [SMILES: *NC(C)(C)C(*)=O]

unless stated otherwise.

“Subject” refers to a human or non-human subject, including, but not limited to, mice, rats, rabbits, dogs, cats, pigs, and non-human primates, including, but not limited to, monkeys and chimpanzees.

“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types. In some embodiments, the cell targeting moiety is an AGTR1 binding cell-targeting moiety.

As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the bridge is in the S configuration. A cEt bicyclic sugar moiety is in the β-D configuration.

“Chemical modification” in a compound describes the substitutions or changes through chemical reaction, of any of the units in the compound. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, a subject, or a human.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to an oligonucleotide. Conjugate groups include a conjugate moiety and a conjugate linker that attaches the conjugate moiety to the oligonucleotide.

“Conjugate linker” means a single bond or a group of atoms comprising at least one bond that connects a conjugate moiety to an oligonucleotide.

“Conjugate moiety” means a group of atoms that is attached to an oligonucleotide via a conjugate linker. In some embodiments, the conjugate moiety comprises an AGTR1 binding cell-targeting moiety. In some embodiments, the conjugate moiety comprises a cell-targeting moiety and a peptide extender. In some embodiments, the conjugate moiety comprises an AGTR1 binding cell-targeting moiety and a peptide extender.

“Designing” or “designed to” refer to the process of designing a compound that specifically hybridizes with a selected nucleic acid molecule.

“Differently modified” means chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Double-stranded antisense compound” means an antisense compound comprising two oligomeric compounds that are complementary to each other and form a duplex, and wherein one of the two said oligomeric compounds comprises an oligonucleotide.

“Expression” includes all the functions by which a gene's coded information is converted into structures present and operating in a cell. Such structures include, but are not limited to, the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Inhibiting the expression or activity” refers to a reduction or blockade of the expression or activity relative to the expression of activity in an untreated or control sample, and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Linked nucleosides” means adjacent nucleosides linked together by an internucleoside linkage.

“Linker-nucleoside” means a nucleoside that links an oligonucleotide to a conjugate moiety. Linker-nucleosides are located within the conjugate linker of a compound. Linker-nucleosides are not considered part of the oligonucleotide portion of a compound even if they are contiguous with the oligonucleotide.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating target nucleic acid can mean to increase or decrease the level of target nucleic acid in a cell, tissue, organ or organism. A “modulator” effects the change in the cell, tissue, organ or organism. For example, a compound can be a modulator that decreases the amount of target nucleic acid in a cell, tissue, organ or organism.

“MOE” means methoxyethyl.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring. “Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Peptide extender” means a peptide that extends from a cell-targeting moiety via an amide bond and attaches to an oligonucleotide via a conjugate linker. In certain embodiments, the cell-targeting moiety comprises or consists of an AGTR1 binding cell-targeting moiety, and the peptide extender extends from the AGTR1 binding cell-targeting moiety. In certain embodiments, a conjugate moiety comprises or consists of a peptide extender and a cell-targeting moiety.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Reduce” means to bring down to a smaller extent, size, amount, or number.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Selective” with respect to an effect refers to a greater effect on one thing over another by any quantitative extent or fold-difference. For example, a compound comprising a AGTR1 conjugate ligand moiety (or AGTR1 binding cell-targeting moiety) that is “selective” for cells expressing AGTR1 or “selectively” targets cells expressing AGTR1, targets cells expressing AGTR1 to a greater extent than a compound not comprising a AGTR1 conjugate ligand moiety. As another example, a compound comprising a AGTR1 conjugate ligand moiety that is “selective” for cells expressing AGTR1 receptor or “selectively” targets cells expressing AGTR1, targets cells expressing AGTR1 to a greater extent than cells that do not express or express relatively lower levels of AGTR1. It will be understood that the term “selective” does not require absolute all-or-none selectivity.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites” are defined as unique nucleobase positions within a target nucleic acid.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” with reference to a target nucleic acid means to reduce or block expression of the target nucleic acid while exhibiting fewer, minimal, or no effects on non-target nucleic acids. Reduction does not necessarily indicate a total elimination of the target nucleic acid's expression.

“Standard cell assay” means assay(s) described in the Examples and reasonable variations thereof.

“Standard in vivo experiment” means the procedure(s) described in the Example(s) and reasonable variations thereof.

“Sugar moiety” means an unmodified sugar moiety or a modified sugar moiety. “Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary compounds or nucleic acids.

“Target gene” refers to a gene encoding a target.

“Targeting” with respect to a target nucleic acid means the specific hybridization of an oligonucleotide to said target nucleic acid in order to induce a desired effect. “Targeting” with respect to a AGTR1 means binding of a AGTR1 binding cell-targeting moiety to AGTR1.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

“Target segment” means the sequence of nucleotides of a target nucleic acid to which a compound is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

### CERTAIN EMBODIMENTS

The present disclosure provides the following non-limiting numbered embodiments:

### Embodiment 1

A compound comprising a modified oligonucleotide linked to an angiotensin II type I receptor (AGTR1) binding conjugate moiety.

### Embodiment 2

The compound of embodiment 1, wherein the modified oligonucleotide is linked to the AGTR1 binding conjugate moiety via a conjugate linker.

### Embodiment 3

The compound of embodiment 1, wherein the AGTR1 binding conjugate moiety comprises a AGTR1 cell-targeting moiety.

### Embodiment 4

The compound of embodiment 1, wherein the AGTR1 binding conjugate moiety consists of a AGTR1 cell-targeting moiety.

### Embodiment 5

The compound of embodiment 1, wherein the AGTR1 binding conjugate moiety comprises an AGTR1 cell-targeting moiety and a peptide extender.

### Embodiment 6

A compound comprising,
a) a modified oligonucleotide; and
b) a conjugate group comprising a conjugate moiety and a conjugate linker,
wherein the conjugate moiety comprises an AGTR1 binding cell-targeting moiety and a peptide extender, and
wherein the conjugate linker links the conjugate moiety to the oligonucleotide via the peptide extender.

### Embodiment 7

The compound of any of embodiments 1-6, wherein the AGTR1 binding cell-targeting moiety is a peptide cell-targeting moiety, small molecule cell-targeting moiety, aptamer cell-targeting moiety, or antibody cell-targeting moiety targeted to AGTR1.

### Embodiment 8

The compound of any of embodiments 1-6, wherein the AGTR1 binding cell-targeting moiety comprises a peptide represented by amino acid sequence A₁A₂A₃A₄A₅A₆A₇A₈ (SEQ ID NO: 11), wherein
A₁ is selected from Asp, Sar, Ala, and NH₂;
A₂ is selected from Arg and Gln;
A₃ is selected from Val and Ala;
A₄ is selected from Tyr, Ala, Ile, Gly, Cha;
A₅ is selected from Ile and Val;
A₆ is selected from His and Ala;
A₇ is selected from Pro and Ala; and
A₈ is selected from Phe, Ala, Ile, Gly, Cha, and Dip,
wherein Sar is N-methylglycine, Cha is beta-cyclohexylalanine, and Dip is diphenylalanine.

### Embodiment 9

The compound of embodiment 8, wherein A₁A₂A₃A₄A₅A₆A₇A₈ (SEQ ID NO: 11) is Asp-Arg-Val-Tyr-Ile-His-Pro-Phe (SEQ ID NO: 12).

### Embodiment 10

The compound of embodiment 8, wherein A₁A₂A₃A₄A₅A₆A₇A₈ (SEQ ID NO: 11) is Asp-Arg-Val-Tyr-Val-His-Pro-Phe (SEQ ID NO: 13).

### Embodiment 11

The compound of any of embodiments 2-10, wherein the modified oligonucleotide is attached to the cell-targeting moiety or the peptide extender via click chemistry, via a disulfide bridge, or via a maleimide linker.

### Embodiment 12

The compound of any of embodiments 2-11, wherein the conjugate linker is selected from the group consisting of pyrrolidine, 8-amino-3,6-dioxaoctanoic acid (ADO), succinimidyl 4-(N-maleimidomethyl) cyclohexane-1-carboxylate (SMCC), and 6-aminohexanoic acid.

### Embodiment 13

The compound of any of embodiments 2-11, wherein the conjugate linker is (p)-6-aminohexanol-1-carboxymethyl[triazoloBCN1]carbamate.

### Embodiment 14

The compound of any of embodiments 1-13, wherein the AGTR1 binding cell-targeting moiety is a peptide cell-targeting moiety comprising a modified lysine that links the peptide cell-targeting moiety to the conjugate linker.

### Embodiment 15

The compound of embodiment 14, wherein the modified lysine is linked to the amino terminus of A₁.

### Embodiment 16

The compound of embodiment 14, wherein the modified lysine is linked to the carboxy terminus of A₈.

### Embodiment 17

The compound of embodiment 14, wherein the modified lysine comprises an azide.

### Embodiment 18

The compound of embodiment 14, wherein the modified lysine is azido-acetyl-lysine.

### Embodiment 19

The compound of any of embodiments 6-18, wherein the conjugate group comprises the sequence selected from [N6-(2-azidoacetyl)-K]DRVYIHPF (SEQ ID NO: 14), [N6-(2-azidoacetyl)-K]PPPAGSSPGDRVYIHPF (SEQ ID NO: 15), XDRVYIHPF (SEQ ID NO: 16), and XPPPAGSSPGDRVYIHPF (SEQ ID NO: 17), wherein X is selected from lysine, D-lysine, L-lysine, and N6-(2-azidoacetyl)-lysine.

### Embodiment 20

The compound of any one of embodiments 6-18, wherein the peptide extender comprises or consists essentially of 3 to 50, 3 to 45, 3 to 40, 3 to 35, 3 to 30, 3 to 25, 3 to 20, 3 to 15, 3 to 10, 6 to 50, 6 to 45, 6 to 40, 6 to 35, 6 to 30, 6 to 25, 6 to 20, 6 to 15, or 6 to 10 amino acids.

### Embodiment 21

The compound of any one of embodiments 6-18, wherein the peptide extender comprises at least 6, at least 7, at least 8, at least 9, or at least 10 amino acids.

### Embodiment 22

The compound of any one of embodiments 6-18, wherein the peptide extender comprises at least one, at least 2 or at least 3 amino acids selected from serine, proline, hydroxyproline, methionine, cysteine and tyrosine.

### Embodiment 23

The compound of embodiment 22, wherein the at least 2 or at least 3 amino acids are contiguous.

### Embodiment 24

The compound of any one of embodiments 6-18, wherein the peptide extender comprises three contiguous proline residues.

### Embodiment 25

The compound of any one of embodiments 6-18, wherein the peptide extender has a molecular weight of about 400 g/mol to about 1800 g/mol.

### Embodiment 26

The compound of any one of embodiments 2-25, wherein the conjugate linker is connected to the 5′ end of the oligonucleotide.

### Embodiment 27

The compound of any one of embodiments 2-25, wherein the conjugate linker is connected to the 3′ end of the oligonucleotide.

### Embodiment 28

The compound of any one of embodiments 2-25, wherein the conjugate linker is connected to a carboxy terminus of the peptide extender.

### Embodiment 29

The compound of any one of embodiments 2-25, wherein the conjugate linker is connected to an amino terminus of the peptide extender.

### Embodiment 30

The compound of any one of embodiments 5-29, wherein the peptide extender has an amino acid sequence selected from: X₁PPPAGSSPG (SEQ ID NO: 30), X₂PPPAGSSPG (SEQ ID NO: 31), X₁ X₂PPAGSSPG (SEQ ID NO: 32), X₁P X₂PAGSSPG (SEQ ID NO: 33), X₁PP X₂AGSSPG (SEQ ID NO: 34), X₁PPP X₂GSSPG (SEQ ID NO: 35), X₁PPPA X₂SSPG (SEQ ID NO: 36), X₁PPPAG X₂SPG (SEQ ID NO: 37), X₁PPPAGS X₂PG (SEQ ID NO: 38), X₁PPPAGSS X₂G (SEQ ID NO: 39), and X₁PPPAGSSP X₂ (SEQ ID NO: 40), wherein X₁ is selected from selected from lysine, D-lysine, L-lysine, and N6-(2-azidoacetyl)-lysine and X₂ is any amino acid.

### Embodiment 31

The compound of any one of embodiments 5-29, wherein the peptide extender has an amino acid sequence that is at least 70%, at least 75%, at least 80%, at least 85%, at least 90%, at least 95%, or 100% identical to the amino acid sequence of X₁PPPAGSSPG (SEQ ID NO: 30), wherein X₁ is selected from selected from lysine, D-lysine, L-lysine, and N6-(2-azidoacetyl)-lysine.

### Embodiment 32

The compound of any one of embodiments 5-29, wherein the peptide extender has an amino acid sequence selected from: CPPPAGSSPG (SEQ ID NO: 41), XPPPAGSSPG (SEQ ID NO: 31), CXPPAGSSPG (SEQ ID NO: 42), CPXPAGSSPG (SEQ ID NO: 43), CPPXAGSSPG (SEQ ID NO: 44), CPPPXGSSPG (SEQ ID NO: 45), CPPPAXSSPG (SEQ ID NO: 46), CPPPAGXSPG (SEQ ID NO: 47), CPPPAGSXPG (SEQ ID NO: 48), CPPPAGSSXG (SEQ ID NO: 49), and CPPPAGSSPX (SEQ ID NO: 50), wherein X is any amino acid.

### Embodiment 33

The compound of any one of embodiments 5-29, wherein the peptide extender comprises an amino acid sequence that is at least 70%, at least 75%, at least 80%, at least 85%, at least 90%, at least 95%, or 100% identical to the amino acid sequence of CPPPAGSSPG (SEQ ID NO: 41).

### Embodiment 34

The compound of any one of embodiments 5-29, wherein the peptide extender is represented by amino acid sequence CAGSIKPPPAGSSPG (SEQ ID NO: 51) or KAGSIKPPPAGSSPG (SEQ ID NO: 52).

### Embodiment 35

The compound of any one of embodiments 5-29, wherein the peptide extender has an amino acid sequence selected from: XPAPSGPSPG (SEQ ID NO: 53), XAGSIKPPPAGSSPG (SEQ ID NO: 54), and XAGMSGASAG (SEQ ID NO: 55), wherein X is selected from lysine, D-lysine, L-lysine, and N6-(2-azidoacetyl)-lysine, and cysteine.

### Embodiment 36

The compound of any one of embodiments 30, 31, or, 34, wherein the lysine of the peptide extender is D-Lysine.

### Embodiment 37

The compound of any one of embodiments 3-31, wherein the peptide extender has a net charge of 0, 1, or 2 at neutral pH.

### Embodiment 38

The compound of any one of embodiments 1-37, wherein the AGTR1 binding conjugate moiety consists or consists essentially of the peptide cell-targeting moiety.

### Embodiment 39

The compound of any of embodiments 1-38, wherein the modified oligonucleotide is 8 to 80 linked nucleosides in length.

### Embodiment 40

The compound of any of embodiments 1-38, wherein the modified oligonucleotide is 10 to 30 linked nucleosides in length.

### Embodiment 41

The compound of any of embodiments 1-38, wherein the modified oligonucleotide is 12 to 30 linked nucleosides in length.

### Embodiment 42

The compound of any of embodiments 1-38, wherein the modified oligonucleotide is 15 to 30 linked nucleosides in length.

### Embodiment 43

The compound of any one of embodiments 1-38, wherein the modified oligonucleotide comprises at least one modified internucleoside linkage, at least one modified sugar, and/or at least one modified nucleobase.

### Embodiment 44

The compound of embodiment 43, wherein the modified internucleoside linkage is a phosphorothioate internucleoside linkage.

### Embodiment 45

The compound of embodiment 44, wherein each internucleoside linkage of the modified oligonucleotide is a phosphorothioate internucleoside linkage.

### Embodiment 46

The compound of embodiment 43, wherein the modified sugar is a bicyclic sugar.

### Embodiment 47

The compound of embodiment 46, wherein the bicyclic sugar is selected from the group consisting of: 4′-(CH2)—O-2′ (LNA); 4′-(CH2)2-O-2′ (ENA); and 4′-CH(CH3)—O-2′ (cEt).

### Embodiment 48

The compound of embodiment 46, wherein the bicyclic sugar is in the β-D configuration.

### Embodiment 49

The compound of embodiment 43, wherein the modified sugar is a non-bicyclic sugar.

### Embodiment 50

The compound of embodiment 49, wherein the non-bicyclic sugar is selected from the group consisting of 2′-O-methoxyethyl, 2′-F, and 2′-OMe.

### Embodiment 51

The compound of embodiment 43, wherein the modified nucleobase is a 5-methylcytosine.

### Embodiment 52

The compound of any one of embodiments 1-51, wherein the modified oligonucleotide comprises:
a) a gap segment consisting of linked deoxynucleosides;
b) a 5′ wing segment consisting of linked nucleosides; and
c) a 3′ wing segment consisting of linked nucleosides;
wherein the gap segment is positioned immediately adjacent to and between the 5′ wing segment and
the 3′ wing segment and wherein each nucleoside of each wing segment comprises a modified sugar.

### Embodiment 53

The compound of any one of embodiments 1-52, wherein the modified oligonucleotide is single-stranded.

### Embodiment 54

The compound of any one of embodiments 1-53, wherein the modified oligonucleotide is an antisense oligonucleotide.

### Embodiment 55

The compound of embodiment 1 or embodiment 6, wherein the modified oligonucleotide is a miRNA antagonist or miRNA mimic.

### Embodiment 56

The compound of embodiment 1 or embodiment 6, wherein the compound comprises a double-stranded duplex.

### Embodiment 57

The compound of embodiment 56, wherein the double-stranded duplex comprises:
a) a first strand comprising the modified oligonucleotide; and
b) a second strand complementary to the first strand.

### Embodiment 58

The compound of embodiment 57, wherein the first strand comprising the modified oligonucleotide is complementary to a RNA transcript.

### Embodiment 59

The compound of embodiment 57 or 58, wherein the second strand is complementary to a RNA transcript.

### Embodiment 60

The compound of embodiment 55, wherein the compound is a miRNA mimic.

### Embodiment 61

The compound of any of embodiments 1-60, wherein the compound comprises at least one ribonucleotide.

### Embodiment 62

The compound of any of embodiments 1-60, wherein the compound comprises at least one β-D-2′-deoxyribosyl sugar moiety.

### Embodiment 63

The compound of any of embodiments 1-62, wherein the modified oligonucleotide is complementary to a RNA transcript.

### Embodiment 64

The compound of embodiment 63, wherein the RNA transcript is pre-mRNA, mRNA, non-coding RNA, or miRNA.

### Embodiment 65

A composition comprising the compound of any one of embodiments 1-64 and a pharmaceutically acceptable carrier or diluent.

### Embodiment 66

A composition consisting or consisting essentially of the compound of any one of embodiments 1-64 and a pharmaceutically acceptable carrier or diluent.

### Embodiment 67

The composition of embodiment 65 or 66, wherein the pharmaceutically acceptable carrier or diluent is phosphate buffered saline (PBS).

### Embodiment 68

The compound of any one of embodiments 1-64, wherein the compound is in a form of a salt.

### Embodiment 69

The compound of embodiment 68, wherein the salt is a sodium salt.

### Embodiment 70

A method of modulating the expression of a nucleic acid target in a cell expressing AGTR1 comprising contacting the cell with the compound or composition of any preceding embodiment, thereby modulating expression of the nucleic acid target in the cell.

### Embodiment 71

The method of embodiment 70, wherein the cell is located on or within a tissue selected from heart, adipose, adrenal gland, liver, and kidney.

### Embodiment 72

The method of embodiment 70 or 71, comprising administering the compound or composition to a subject.

### Embodiment 73

The method of embodiment 72, wherein the subject has a condition or disease of a tissue selected from heart, adipose, adrenal gland, liver, and kidney.

### Embodiment 74

The method of embodiment 73, wherein the subject is at risk of a condition or disease of a tissue selected from heart, adipose, adrenal gland, liver, and kidney.

### Embodiment 75

The method of any of embodiments 70-74, wherein the compound inhibits expression of the nucleic acid target.

### Certain Compounds Comprising an Oligonucleotide

In certain embodiments, provided herein are oligomeric compounds comprising an oligonucleotide and an AGTR1 binding cell-targeting moiety. In certain embodiments, the oligonucleotide is a modified oligonucleotide. In certain embodiments, the oligonucleotide is an unmodified oligonucleotide. In certain embodiments, oligomeric compounds comprise an oligonucleotide, an AGTR1 binding cell-targeting moiety, a peptide extender, and a conjugate linker. In certain embodiments, the conjugate linker connects the peptide extender to the oligonucleotide, and the peptide extender connects the conjugate linker to the AGTR1 binding cell-targeting moiety. In certain embodiments, the oligonucleotide and the AGTR1 binding cell-targeting moiety are connected via the peptide extender and a conjugate linker, wherein the oligonucleotide is directly connected to the conjugate linker, the conjugate linker is directly connected to the peptide extender and the peptide extender is directly connected to the AGTR1 binding cell-targeting moiety.

In certain embodiments, compounds described herein can be antisense compounds. In certain embodiments, the antisense compound comprises or consists of an oligomeric compound. In certain embodiments, the oligomeric compound comprises a oligonucleotide, such as a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide and optionally a conjugate group. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, compounds are double-stranded. Such double-stranded compounds comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, compound comprises a conjugate group. In certain embodiments, one of the modified oligonucleotides is conjugated. In certain embodiments, both the modified oligonucleotides are conjugated. In certain embodiments, the first modified oligonucleotide is conjugated. In certain embodiments, the second modified oligonucleotide is conjugated. In certain embodiments, the first modified oligonucleotide is 12-30 linked nucleosides in length and the second modified oligonucleotide is 12-30 linked nucleosides in length. In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In certain embodiments, a compound comprises a double-stranded duplex comprising (i) a first strand comprising a modified oligonucleotide, optionally a conjugate linker, and a AGTR1 binding cell-targeting moiety, and (ii) a second strand complementary to the first strand. In certain embodiments, a compound comprises a double-stranded duplex comprising (i) a first strand comprising the modified oligonucleotide, optionally a conjugate linker, and a AGTR1 binding cell-targeting moiety, and (ii) a second strand complementary to the first strand; wherein the first strand is complementary to a RNA transcript. In certain embodiments, a compound comprises a double-stranded duplex comprising (i) a first strand comprising a modified oligonucleotide, optionally a conjugate linker, and a AGTR1 binding cell-targeting moiety, and (ii) a second strand complementary to the first strand; wherein the second strand is complementary to a RNA transcript.

Examples of single-stranded and double-stranded compounds include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 10 to 30 linked subunits in length. In certain embodiments, a compound described herein comprises an oligonucleotide 12 to 30 linked subunits in length. In certain embodiments, a compound described herein comprises an oligonucleotide 12 to 22 linked subunits in length. In certain embodiments, compound described herein comprises an oligonucleotide 14 to 30 linked subunits in length. In certain embodiments, compound described herein comprises an oligonucleotide 14 to 20 linked subunits in length. In certain embodiments, a compound described herein comprises an oligonucleotide 15 to 30 linked subunits in length. In certain embodiments, a compound described herein comprises an oligonucleotide 15 to 20 linked subunits in length. In certain embodiments, a compound described herein comprises an oligonucleotide 16 to 30 linked subunits in length. In certain embodiments, a compound described herein comprises an oligonucleotide 16 to 20 linked subunits in length. In certain embodiments, a compound described herein comprises an oligonucleotide 17 to 30 linked subunits in length. In certain embodiments, a compound described herein comprises an oligonucleotide 17 to 20 linked subunits in length. In certain embodiments, a compound described herein comprises an oligonucleotide 18 to 30 linked subunits in length. In certain embodiments, a compound described herein comprises an oligonucleotide 18 to 21 linked subunits in length. In certain embodiments, a compound described herein comprises an oligonucleotide 18 to 20 linked subunits in length. In certain embodiments, a compound described herein comprises an oligonucleotide 20 to 30 linked subunits in length. In other words, such oligonucleotides are 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits in length, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the linked subunits are nucleotides, nucleosides, or nucleobases.

In certain embodiments, the compound may further comprise additional features or elements, such as a conjugate group, that are attached to the oligonucleotide. In certain embodiments, such compounds are antisense compounds. In certain embodiments, such compounds are oligomeric compounds. In embodiments where a conjugate group comprises a nucleoside (i.e. a nucleoside that links the conjugate group to the oligonucleotide), the nucleoside of the conjugate group is not counted in the length of the oligonucleotide.

In certain embodiments, compounds may be shortened or truncated. For example, a single subunit may be deleted from the 5′ end (5′ truncation), or alternatively from the 3′ end (3′ truncation). A shortened or truncated compound targeted to a nucleic acid may have two subunits deleted from the 5′ end, or alternatively may have two subunits deleted from the 3′ end, of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively to the 3′ end (3′ addition), of the compound. Alternatively, the added subunits may be dispersed throughout the compound.

It is possible to increase or decrease the length of a compound, such as an oligonucleotide, and/or introduce mismatch bases without eliminating activity (Woolf et al. (Proc. Natl. Acad. Sci. USA 89:7305-7309, 1992; Gautschi et al. J. Natl. Cancer Inst. 93:463-471, March 2001; Maher and Dolnick Nuc. Acid. Res. 16:3341-3358,1988). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term “RNAi” is meant to be equivalent to other terms used to describe sequence specific RNA interference, such as post transcriptional gene silencing, translational inhibition, or epigenetics.

In certain embodiments, the first strand of the compound is an siRNA guide strand and the second strand of the compound is an siRNA passenger strand. In certain embodiments, the second strand of the compound is complementary to the first strand. In certain embodiments, each strand of the compound is 16, 17, 18, 19, 20, 21, 22, or 23 linked nucleosides in length. In certain embodiments, the first or second strand of the compound can comprise a conjugate group.

In certain embodiments, compounds described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the compound that are beneficial for use as a therapeutic or research tool. In certain embodiments, radioactive isotopic substitutions may make the compound suitable for research or diagnostic purposes, such as an imaging assay.

### Certain Mechanisms

In certain embodiments, compounds described herein comprise or consist of modified oligonucleotides. In certain embodiments, compounds described herein are antisense compounds. In certain embodiments, compounds comprise oligomeric compounds. In certain embodiments, compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or subject.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid is a non-coding RNA. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

### Hybridization

In some embodiments, hybridization occurs between a compound disclosed herein and a target nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with a target nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a target nucleic acid may be tolerated provided that the compound remains able to specifically hybridize to a target nucleic acid. Moreover, a compound may hybridize over one or more segments of a target nucleic acid such that intervening or adjacent segments are not involved in the hybridization event (e.g., a loop structure, mismatch or hairpin structure).

In certain embodiments, the compounds provided herein, or a specified portion thereof, are, are at least, or are up to 70%, 80%, 85%, 86%, 87%, 88%, 89%, 90%, 91%, 92%, 93%, 94%, 95%, 96%, 97%, 98%, 99%, or 100% complementary to a target nucleic acid, a target region, target segment, or specified portion thereof. In certain embodiments, the compounds provided herein, or a specified portion thereof, are 70% to 75%, 75% to 80%, 80% to 85%, 85% to 90%, 90% to 95%, 95% to 100%, or any number in between these ranges, complementary to a target nucleic acid, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid. Percent complementarity of a compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be fully complementary to a target nucleic acid, or a target region, or a target segment or target sequence thereof. As used herein, “fully complementary” means each nucleobase of a compound is complementary to the corresponding nucleobase of a target nucleic acid. For example, a 20 nucleobase compound is fully complementary to a target sequence that is 400 nucleobases long, so long as there is a corresponding 20 nucleobase portion of the target nucleic acid that is fully complementary to the compound. Fully complementary can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 1, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a target nucleic acid, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a target nucleic acid, or specified portion thereof.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the—compounds, are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 11, 12, 13, 14, 15, 16, 17, 18, 19, 20, or more nucleobase portion of a target segment, or a range defined by any two of these values.

### Identity

The compounds provided herein may also have a defined percent identity to a particular nucleotide sequence, SEQ ID NO, or compound represented by a specific Compound number, or portion thereof. In certain embodiments, compounds described herein are antisense compounds or oligomeric compounds. In certain embodiments, compounds described herein are modified oligonucleotides. As used herein, a compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of an compound is calculated according to the number of bases that have identical base pairing relative to the sequence to which it is being compared.

In certain embodiments, compounds described herein, or portions thereof, are, are at least, or are up to 70%, 75%, 80%, 85%, 90%, 91%, 92%, 93%, 94%, 95%, 96%, 97%, 98%, 99% or 100% identical to one or more of the compounds or SEQ ID NOs, or a portion thereof, disclosed herein. In certain embodiments, compounds described herein are about 70%, 75%, 80%, 85%, 90%, 91%, 92%, 93%, 94%, 95%, 96%, 97%, 98%, or 99% identical, or any percentage between such values, to a particular nucleotide sequence, SEQ ID NO, or compound represented by a specific Compound number, or portion thereof, in which the compounds comprise an oligonucleotide having one or more mismatched nucleobases. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 1, 2, 3, 4, 5, 6, 7, 8, 9, 10, 11, or 12 from the 3′-end of the oligonucleotide.

In certain embodiments, compounds described herein comprise or consist of antisense compounds. In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, compounds described herein comprise or consist of oligonucleotides. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Modified Nucleosides

Modified nucleosides comprise a modified sugar moiety or a modified nucleobase or both a modified sugar moiety and a modified nucleobase.

1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)2OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearly non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₘ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃.

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′—C(RₐR_{b})—O—N(RₐR_{b})-2′, 4′-CH₂—O—N(R)-2′, and 4′—CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 91999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. patent publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

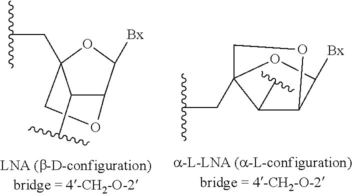

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see e.g., Leumann, C J. Bioorg. & Med. Chem. 2002, 10, 841-854), fluoro HNA:

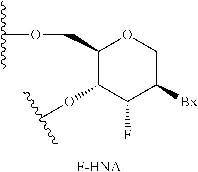

(“F—HNA”, see e.g., Swayze et al., U.S. Pat. No. 8,088,904; Swayze et al., U.S. Pat. No. 8,440,803; Swayze et al., U.S.; and Swayze et al., U.S. Pat. No. 9,005,906, F—HNA can also be referred to as a F-THP or 3′-fluoro tetrahydropyran), and nucleosides comprising additional modified THP compounds having the formula:

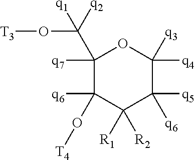

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5 or Y-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

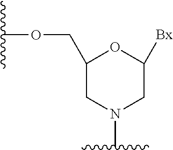

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds.

In certain embodiments, compounds described herein comprise modified oligonucleotides. In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi-dines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH3) uracil, 5-propynylcytosine, 6-azouracil, 6-azacytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds targeted to a target nucleic acid comprise one or more modified nucleobases. In certain embodiments, the modified nucleobase is 5-methylcytosine. In certain embodiments, each cytosine is a 5-methylcytosine.

3. Modified Internucleoside Linkages

The naturally occurring internucleoside linkage of RNA and DNA is a 3 to 5′ phosphodiester linkage In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

In certain embodiments, compounds targeted to a target nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH2-N(CH3)—O—CH2-), thiodiester, thionocarbamate (—O—C(═O)(NH)—S—); siloxane (—O—SiH2—O—); and N,N′-dimethylhydrazine (—CH2—N(CH3)—N(CH3)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH2-N(CH3)—O-5′), amide-3 (3′-CH2-C(═O)—N(H)-5′), amide-4 (3′-CH2—N(H)—C(═O)-5′), formacetal (3′-O—CH2—O-5′), methoxypropyl, and thioformacetal (3′-S—CH2—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, oligonucleotides having a gapmer nucleoside motif comprise a linkage motif comprising all phosphorothioate linkages except for one or two methylphosphonate linkages. In certain embodiments, one methylphosphonate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

4. Certain Motifs

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5-wing differs from the sugar motif of the Y-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

5. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are characterized by their modification, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

### Certain Conjugated Compounds

In certain embodiments, the compounds described herein comprise or consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### AGTR1 Binding Conjugate Moieties

In certain embodiments, a compound comprises an oligonucleotide and AGTR1 binding conjugate moiety. In certain embodiments, a compound comprises an oligonucleotide, conjugate linker, and a AGTR1 binding cell-targeting moiety. In certain embodiments, the conjugate linker links the AGTR1 binding cell-targeting moiety to the oligonucleotide. In certain embodiments, the oligonucleotide is a modified oligonucleotide. In certain embodiments, the AGTR1 binding cell-targeting moiety comprises a small molecule, aptamer, antibody, or peptide.

1. Certain AGTR1 Binding Small Molecule Conjugate Moieties

In certain embodiments, a compound comprises an oligonucleotide and a small molecule conjugate moiety capable of binding to AGTR1. In certain embodiments, a compound comprises an oligonucleotide, conjugate linker, and small molecule conjugate moiety capable of binding to AGTR1. In certain embodiments, the oligonucleotide is a modified oligonucleotide.

Any small molecule conjugate moiety capable of binding to AGTR1 known in the art can be used in several embodiments. For example, in certain embodiments the small molecule conjugate moiety capable of binding to AGTR1 is a small molecule AGTR1 antagonist described in Willard et al., “Small Molecule Drug Discovery at the Glucagon-like Peptide-1 Receptor,” Experimental Diabetes Research Vol. 2012 pgs. 1-9; Sloop et al., “Novel Small Molecule Glucagon-Like Peptide-1 Receptor Agonist Stimulates Insulin Secretion in Rodents and From Human Islets,” Diabetes Vol, 59, 2010 pgs. 3099-3107; Knudsen et al., “Small-molecule agonists for the glucagon-like peptide 1 receptor,” PNAS Jan. 16, 2007; 104(3):937-42; or Wang et al., “Non-peptidic glucose-like peptide-1 receptor agonists: aftermath of a serendipitous discovery,” Acta Pharmacologica Sinica (2010) 31: 1026-1030; which are incorporated by reference herein in their entireties.

2. Certain AGTR1 Antibody Conjugate Moieties

In certain embodiments, a compound comprises an oligonucleotide and an antibody or fragment thereof capable of binding to AGTR1. In certain embodiments, a compound comprises an oligonucleotide, conjugate linker, and an antibody or fragment thereof capable of binding to AGTR1. In certain embodiments, the oligonucleotide is a modified oligonucleotide. Any antibody or fragment thereof capable of binding to AGTR1 known in the art can be used in several embodiments.

3. Certain AGTR1 Peptide Conjugate Moieties

In certain embodiments, compounds disclosed herein comprise a peptide capable of binding AGTR1, also referred to herein as an AGTR1 peptide conjugate moiety. In certain embodiments, the AGTR1 peptide conjugate moiety comprises an Ang II peptide hormone. In certain embodiments, the AGTR1 peptide conjugate moiety consists of or consists essentially of an Ang II peptide hormone. In certain embodiments, the AGTR1 peptide conjugate moiety comprises an analog of an Ang II peptide hormone (Ang II analog) that is capable of binding AGTR1. In certain embodiments, the AGTR1 peptide conjugate moiety consists or consists essentially of an analog of an Ang II peptide hormone (Ang II analog) that is capable of binding AGTR1. Ang II analogs are known in the art. For example, Ang II analogs are described by Holloway et al., (Molecular Pharmacology 61:768-777 (2002)). In certain embodiments, the AGTR1 peptide conjugate moiety is capable of binding AGTR1, wherein binding results cellular internalization of AGTR1. In certain embodiments, the AGTR1 peptide conjugate moiety is represented by the amino acid sequence A₁A₂A₃A₄A₅A₆A₇A₈, (SEQ ID NO: 11) wherein A₁ is selected from Asp, Sar, Ala, and NH2; A₂ is selected from Arg and Gln; A₃ is selected from Val and Ala; A₄ is selected from Tyr, Ala, Ile, Gly, Cha; A₅ is selected from Ile and Val; A₆ is selected from His and Ala; A₇ is selected from Pro and Ala; and A₈ is selected from Phe, Ala, Ile, Gly, Cha, and Dip, wherein Sar is N-methylglycine, Cha is beta-cyclohexylalanine, and Dip is diphenylalanine. In certain embodiments, the AGTR1 peptide conjugate moiety is represented by the amino acid sequence Asp-Arg-Val-Tyr-Ile-His-Pro-Phe (SEQ ID NO: 12). In certain embodiments, the AGTR1 peptide conjugate moiety is represented by amino acid sequence Asp-Arg-Val-Tyr-Val-His-Pro-Phe (SEQ ID NO: 13).

In certain embodiments, the AGTR1 peptide conjugate moiety comprises a lysine at its amino terminus. In certain embodiments, the AGTR1 peptide conjugate moiety comprises a lysine at its carboxy terminus. In certain embodiments, the lysine is directly linked to the modified oligonucleotide. In certain embodiments, the lysine is indirectly linked to the modified oligonucleotide. For example, the compound may comprise a conjugate linker, wherein the lysine is directly linked to the conjugate linker at a first point on the conjugate linker and the modified oligonucleotide is directly linked to the conjugate linker at a second point on the conjugate linker, wherein the first point and the second point are different. In some embodiments, an amino terminal lysine of the AGTR1 peptide conjugate moiety is modified to azido-acetyl lysine to facilitate the conjugation of the conjugate moiety to the modified oligonucleotide. The azido-acetyl group is attached to the side chain amine as shown below:

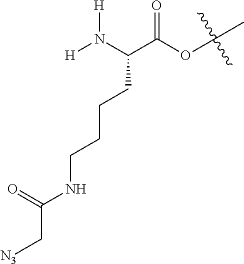 [SMILES: *OC(=O)[C@@H](N)CCCCNC(=O)CN=[N+]=[N-]]

In some embodiments, the AGTR1 peptide conjugate moiety comprises the sequence of [N6-(2-azidoacetyl)-K]DRVYIHPF (SEQ ID NO: 14). In some embodiments, the AGTR1 peptide conjugate moiety comprises the sequence of XDRVYIHPF (SEQ ID NO: 16), wherein X is selected from lysine, D-lysine, L-lysine, and N6-(2-azidoacetyl)-lysine

In certain embodiments, compounds disclosed herein comprise a modified oligonucleotide and a AGTR1 peptide conjugate moiety comprising an amino acid sequence with 1, 2, 3, 4, 5, 6, 7 or 8 amino acid substitutions, insertions, deletions, or a combination of two or more thereof, when compared to the amino acid sequence of Asp-Arg-Val-Tyr-Ile-His-Pro-Phe (SEQ ID NO: 12) or Asp-Arg-Val-Tyr-Val-His-Pro-Phe (SEQ ID NO: 13). In certain embodiments, the AGTR1 peptide conjugate moiety comprises a conservative amino acid substitution, an amino acid analog, or an amino acid derivative, when compared to the amino acid sequence of Asp-Arg-Val-Tyr-Ile-His-Pro-Phe (SEQ ID NO: 12) or Asp-Arg-Val-Tyr-Val-His-Pro-Phe (SEQ ID NO: 13). In certain embodiments, the conservative amino acid substitution comprises replacement of an aliphatic amino acid with another aliphatic amino acid; replacement of a serine with a threonine or vice versa; replacement of an acidic residue with another acidic residue; replacement of a residue bearing an amide group with another residue bearing an amide group; exchange of a basic residue with another basic residue; or, replacement of an aromatic residue with another aromatic residue, or a combination thereof, and the aliphatic residue comprises Alanine, Valine, Leucine, Isoleucine or a synthetic equivalent thereof; the acidic residue comprises Aspartic acid, Glutamic acid or a synthetic equivalent thereof; the residue comprising an amide group comprises Aspartic acid, Glutamic acid or a synthetic equivalent thereof; the basic residue comprises Lysine, Arginine or a synthetic equivalent thereof; or, the aromatic residue comprises Phenylalanine, Tyrosine or a synthetic equivalent thereof.

### Certain Peptide Extenders

In certain embodiments, oligomeric compounds comprise a peptide extender. In certain embodiments, the peptide extender is capable of providing a distance or barrier between the oligonucleotide and the AGTR1 binding cell-targeting moiety such that the oligonucleotide does not inhibit an activity of the AGTR1 binding cell-targeting moiety, and the AGTR1 binding cell-targeting moiety does not inhibit an activity of the oligonucleotide. In certain embodiments, the peptide extender is capable of providing a distance or barrier between the oligonucleotide and the AGTR1 binding cell-targeting moiety such that the oligonucleotide inhibits an activity of the AGTR1 binding cell-targeting moiety to a lesser degree and/or the AGTR1 binding cell-targeting moiety inhibits an activity of the oligonucleotide to a lesser degree relative to the respective inhibition that would occur in the absence of the peptide extender. In certain embodiments, the activity of the AGTR1 binding cell-targeting moiety is binding a cell surface moiety (e.g., cell surface receptor). In certain embodiments, the activity of the oligonucleotide is an antisense activity.

In general, the peptide extender is not a AGTR1 binding cell-targeting moiety. In certain embodiments, the peptide extender does not interact with a cell-surface moiety. In certain embodiments, the AGTR1 binding cell-targeting moiety and the peptide extender are not peptides encoded by the same species. In certain embodiments, the peptide extender is not a peptide encoded by a human gene.

In certain embodiments, the peptide extender comprises at least one amino acid selected from serine, proline, hydroxyproline, methionine, cysteine and tyrosine. In certain embodiments, the peptide extender comprises at least two, at least three or at least four amino acids selected from serine, proline, hydroxyproline, methionine, cysteine and tyrosine. In certain embodiments, the peptide extender comprises two contiguous amino acids selected from a serine, proline, hydroxyproline, methionine, cysteine and tyrosine. In certain embodiments, the peptide extender comprises three contiguous amino acids selected from a serine, proline, hydroxyproline, methionine, cysteine and tyrosine. In certain embodiments, the peptide extender comprises four contiguous amino acids selected from a serine, proline, hydroxyproline, methionine, cysteine and tyrosine. In certain embodiments, the peptide extender comprises a polyproline helix.

In certain embodiments, the peptide extender does not comprise more than 1 basic amino acid. In certain embodiments, the peptide extender does not comprise any basic amino acids. In certain embodiments, the peptide extender does not comprise more than one lysine or arginine. In certain embodiments, the peptide extender does not comprise a lysine or an arginine. In certain embodiments, the net charge of the peptide extender at pH=7 is less than or equal to 2.

In certain embodiments, the peptide extender has a molecular weight of about 400 g/mol to about 1800 g/mol, about 500 g/mol to about 1700 g/mol, about 600 g/mol to about 1600 g/mol, about 700 g/mol to about 1500 g/mol, or about 800 g/mol to about 1400 g/mol.

In certain embodiments, the peptide extender has a molecular weight of at least about 400 g/mol, at least about 425 g/mol, at least about 450 g/mol, at least about 475 g/mol, at least about 500 g/mol, at least about 525 g/mol, at least about 550 g/mol, at least about 575 g/mol, at least about 600 g/mol, at least about 625 g/mol, at least about 650 g/mol, at least about 675 g/mol, at least about 700 g/mol, at least about 725 g/mol, at least about 750 g/mol, at least about 775 g/mol, at least about 800 g/mol, at least about 825 g/mol, at least about 850 g/mol, at least about 875 g/mol, or at least about 900 g/mol.

In certain embodiments, the peptide extender has a molecular weight of about 400 g/mol, about 425 g/mol, about 450 g/mol, about 475 g/mol, about 500 g/mol, about 525 g/mol, about 550 g/mol, about 575 g/mol, about 600 g/mol, about 625 g/mol, about 650 g/mol, about 675 g/mol, about 700 g/mol, about 725 g/mol, about 750 g/mol, about 775 g/mol, about 800 g/mol, about 825 g/mol, about 850 g/mol, about 875 g/mol, about 900 g/mol, about 925 g/mol, about 950 g/mol, about 975 g/mol, about 1000 g/mol, about 1025 g/mol, about 1050 g/mol, about 1075 g/mol, about 1100 g/mol, about 1125 g/mol, about 1150 g/mol, about 1175 g/mol, about 1200 g/mol, about 1225 g/mol, about 1250 g/mol, about 1275 g/mol, about 1300 g/mol, about 1325 g/mol, about 1350 g/mol, about 1375 g/mol, about 1400 g/mol, about 1425 g/mol, about 1450 g/mol, about 1475 g/mol, about 1500 g/mol, about 1525 g/mol, about 1550 g/mol, about 1575 g/mol, about 1600 g/mol.

In certain embodiments, the peptide extender has a length selected from about 5 Å to about 10 Å, about 10 Å to about 15 Å, about 15 Å to about 20 Å, and about 20 Å to about 25 Å. In certain embodiments, the peptide extender has a length of at least 2 Å, at least 4 Å, at least 6 Å, at least 8 Å, at least 10 Å, at least 12 Å, at least 14 Å, at least 16 Å, at least 18 Å, at least 20 Å, at least 22 Å, or at least 24 Å. In certain embodiments, the peptide extender has a length selected from 10 Å, 11 Å, 12 Å, 13 Å, 14 Å, 15 Å, 16 Å, 17 Å, 18 Å, 19 Å, and 20 Å. In certain embodiments, the length of a peptide extender of an oligomeric compound is its length when the oligomeric compound is present in a solvent. In certain embodiments, the solvent is water. In certain embodiments, the solvent is a saline solution. In certain embodiments, the solvent is phosphate buffered saline (PBS).

In certain embodiments, the peptide extender comprises at least 1, at least 2, at least 3, at least 4, at least 5, at least 6, at least 7, at least 8, at least 9, at least 10, at least 11, or at least 12 amino acids. In certain embodiments, the peptide extender comprises 3 to 50, 3 to 45, 3 to 40, 3 to 35, 3 to 30, 3 to 25, 3 to 20, 3 to 15, 3 to 10, 6 to 50, 6 to 45, 6 to 40, 6 to 35, 6 to 30, 6 to 25, 6 to 20, 6 to 15, or 6 to 10 amino acids. In certain embodiments, the peptide extender comprises 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids. In certain embodiments, the peptide extender consists of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids. In certain embodiments, the peptide extender comprises 9 amino acids. In certain embodiments, the peptide extender consists of 9 amino acids. In certain embodiments, the peptide extender comprises 10 amino acids. In certain embodiments, the peptide extender consists of 10 amino acids.

In certain embodiments, the amino acid sequence of the peptide extender comprises the amino acid sequence of PPPAGSSPG (SEQ ID NO: 20). In certain embodiments, the amino acid sequence of the peptide extender is PPPAGSSPG (SEQ ID NO: 20). In certain embodiments, the amino acid sequence of the peptide extender comprises an amino acid sequence that is at least 75%, at least 80%, at least 85% identical to the amino acid sequence of PPPAGSSPG (SEQ ID NO: 20).

In certain embodiments, the amino acid sequence of the peptide extender comprises an amino acid sequence selected from: PPPAGSSPG (SEQ ID NO: 20), XPPAGSSPG (SEQ ID NO: 21), PXPAGSSPG (SEQ ID NO: 22), PPXAGSSPG (SEQ ID NO: 23), PPPXGSSPG (SEQ ID NO: 24), PPPAXSSPG (SEQ ID NO: 25), PPPAGXSPG (SEQ ID NO: 26), PPPAGSXPG (SEQ ID NO: 27), PPPAGSSXG (SEQ ID NO: 28), and PPPAGSSPX (SEQ ID NO: 29), wherein X is any amino acid. In certain embodiments, X is a nonpolar amino acid. In certain embodiments, X is a non-charged polar amino acid. In certain embodiments, X is a basic amino acid. In certain embodiments, X is an acidic amino acid. In certain embodiments, at least one serine is replaced with a threonine. In certain embodiments, alanine is replaced with valine, leucine, or isoleucine. In certain embodiments, X is not lysine. In certain embodiments, X is not arginine.

In certain embodiments, the peptide extender comprises a lysine at its amino terminus. In certain embodiments, the peptide extender comprises a lysine at its carboxy terminus. In certain embodiments, the lysine is directly linked to the modified oligonucleotide. In certain embodiments, the lysine is indirectly linked to the modified oligonucleotide. For example, the oligomeric compound may comprise a conjugate linker, wherein the lysine is directly linked to the conjugate linker at a first point on the conjugate linker and the modified oligonucleotide is directly linked to the conjugate linker at a second point on the conjugate linker, wherein the first point and the second point are different. In some embodiments, an amino terminal lysine of the peptide extender is modified to azido-acetyl lysine to facilitate the conjugation of peptide extender to the modified oligonucleotide. The azido-acetyl group is attached to the side chain amine as shown below:

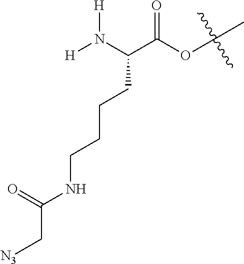 [SMILES: *OC(=O)[C@@H](N)CCCCNC(=O)CN=[N+]=[N-]]

In certain embodiments, the amino acid sequence of the peptide extender comprises or consists of an amino acid sequence selected from: X₁PPPAGSSPG (SEQ ID NO: 30), X₂PPPAGSSPG (SEQ ID NO: 31), X₁X₂PPAGSSPG (SEQ ID NO: 32), X₁PX₂PAGSSPG (SEQ ID NO: 33), X₁PPX₂AGSSPG (SEQ ID NO: 34), X₁PPPX₂GSSPG (SEQ ID NO: 35), X₁PPPAX₂SSPG (SEQ ID NO: 36), X₁PPPAGX₂SPG (SEQ ID NO: 37), X₁PPPAGSX₂PG (SEQ ID NO: 38), X₁PPPAGSSX₂G (SEQ ID NO: 39), and X₁PPPAGSSPX₂ (SEQ ID NO: 40), wherein X₁ is selected from selected from lysine, D-lysine, L-lysine, and N6-(2-azidoacetyl)-lysine and X₂ is any amino acid. In certain embodiments, X is a nonpolar amino acid. In certain embodiments, X is a non-charged polar amino acid. In certain embodiments, X is a basic amino acid. In certain embodiments, X is an acidic amino acid. In certain embodiments, at least one serine is replaced with a threonine. In certain embodiments, alanine is replaced with valine, leucine, or isoleucine. In certain embodiments, X is not lysine. In certain embodiments, X is not arginine. In certain embodiments, the N terminal lysine is selected from D-lysine, L-lysine, N⁶—(2-azidoacetyl)-D-lysine, and N⁶-(2-azidoacetyl)-L-lysine. In certain embodiments, the N terminal lysine is azido-acetyl lysine. In certain embodiments, the amino acid sequence of the peptide extender is at least 75%, at least 80%, or at least 85% identical to an amino acid sequence selected from: X₁PPPAGSSPG (SEQ ID NO: 30), X₂PPPAGSSPG (SEQ ID NO: 31), X₁X₂PPAGSSPG (SEQ ID NO: 32), X₁PX₂PAGSSPG (SEQ ID NO: 33), X₁PPX₂AGSSPG (SEQ ID NO: 34), X₁PPPX₂GSSPG (SEQ ID NO: 35), X₁PPPAX₂SSPG (SEQ ID NO: 36), X₁PPPAGX₂SPG (SEQ ID NO: 37), X₁PPPAGSX₂PG (SEQ ID NO: 38), X₁PPPAGSSX₂G (SEQ ID NO: 39), and X₁PPPAGSSP X₂ (SEQ ID NO: 40), wherein X₁ is selected from selected from lysine, D-lysine, L-lysine, and N6-(2-azidoacetyl)-lysine and X₂ is any amino acid. In certain embodiments, the N terminal lysine is selected from D-lysine, L-lysine, N⁶-(2-azidoacetyl)-D-lysine, and N⁶-(2-azidoacetyl)-L-lysine. In certain embodiments, the N terminal lysine is azido-acetyl lysine.

In certain embodiments, the amino acid of the peptide extender comprises an amino acid sequence of: CPPPAGSSPG (SEQ ID NO: 41). In certain embodiments, the amino acid of the peptide extender consists of an amino acid sequence of: CPPPAGSSPG (SEQ ID NO: 41). In certain embodiments, the peptide extender comprises an amino acid sequence that is at least 75% or at least 85% identical to the amino acid sequence of CPPPAGSSPG (SEQ ID NO: 41).

In certain embodiments, the amino acid sequence of the peptide extender comprises or consists of an amino acid sequence selected from: CPPPAGSSPG (SEQ ID NO: 41), XPPPAGSSPG (SEQ ID NO: 31), CXPPAGSSPG (SEQ ID NO: 42), CPXPAGSSPG (SEQ ID NO: 43), CPPXAGSSPG (SEQ ID NO: 44), CPPPXGSSPG (SEQ ID NO: 45), CPPPAXSSPG (SEQ ID NO: 46), CPPPAGXSPG (SEQ ID NO: 47), CPPPAGSXPG (SEQ ID NO: 48), CPPPAGSSXG (SEQ ID NO: 49), and CPPPAGSSPX (SEQ ID NO: 50) wherein X is any amino acid. In certain embodiments, X is a nonpolar amino acid. In certain embodiments, X is a non-charged polar amino acid. In certain embodiments, X is a basic amino acid. In certain embodiments, X is a acidic amino acid. In certain embodiments, at least one serine is replaced with a threonine. In certain embodiments, alanine is replaced with valine, leucine, or isoleucine. In certain embodiments, X is not lysine. In certain embodiments, X is not arginine.

In certain embodiments, the amino acid sequence of the peptide extender comprises or consists of an amino acid sequence selected from: XPAPSGPSPG (SEQ ID NO: 53), XAGSIKPPPAGSSPG (SEQ ID NO: 54), and XAGMSGASAG (SEQ ID NO: 55), wherein X is selected from lysine, D-lysine, L-lysine, and N6-(2-azidoacetyl)-lysine, and cysteine. In certain embodiments, X is selected from D-lysine, L-lysine, N⁶-(2-azidoacetyl)-D-lysine, and N⁶-(2-azidoacetyl)-L-lysine. In certain embodiments, the amino acid sequence of the peptide extender is at least 75%, at least 80%, at least 85%, or at least 90% identical to an amino acid sequence selected from SEQ ID NOS: 6-8, wherein X is selected from lysine (K) and cysteine (C). In certain embodiments, the amino acid sequence of the peptide extender comprises an amino acid sequence having at least 3, at least 4, at least 5, at least 6, at least 7, or at least 8 consecutive amino acids that are identical to an equal length portion of the amino acid sequence of any one of SEQ ID NOS: 20-50.

In certain embodiments, the peptide extender comprises a linker amino acid that links the peptide extender to the conjugate linker. In certain embodiments, the linker amino acid is selected from lysine, cysteine, azido norleucine, and methionine. For example, the conjugate linker may be formed by click chemistry and the linker amino acid is lysine. Also, by way of example, the conjugate linker may comprise maleimide and the linker amino acid is cysteine. In certain embodiments, the peptide extender comprises the linker amino acid at its amino terminus. In certain embodiments, the peptide extender comprises the linker amino acid at its carboxy terminus. By way of non-limiting example, the peptide extender may be represented by the sequence: X₁PPPAGSSPG (SEQ ID NO: 30), wherein X₁ is selected from selected from lysine, D-lysine, L-lysine, and N6-(2-azidoacetyl)-lysine, wherein “X₁” is the linker amino acid. In certain embodiments, the linker amino acid is D-Lysine, which improves the stability of the peptide as compared to when the linker amino acid is L-Lysine. In certain embodiments, linker amino acid is selected from D-lysine, L-lysine, N⁶-(2-azidoacetyl)-D-lysine, and N⁶-(2-azidoacetyl)-L-lysine. In certain embodiments, X₁ is N6-(2-azidoacetyl)-lysine.

In some embodiments, the AGTR1 conjugate group comprises the sequence of the sequence selected from [N6-(2-azidoacetyl)-K]PPPAGSSPGDRVYIHPF (SEQ ID NO: 15) and XPPPAGSSPGDRVYIHPF (SEQ ID NO: 17), wherein X is selected from lysine, D-lysine, L-lysine, and N6-(2-azidoacetyl)-lysine.

In some embodiments, the peptide extender comprises a structure of the formula X_{L}—(X_{S})ₙ—X₁—X₂—X₃—X₄—X₅—X₆—X₇—X₈—X₉, wherein:

i) X_{L} comprises a linker amino acid;

ii) n is from 0 to 5;

iii) at least one of X_{S} is independently selected from an aliphatic amino acid, lysine, and serine;

iv) at least one of X₁—X₉ is a neutral amino acid.

In some embodiments, each of X₁—X₉ is a neutral amino acid. In some embodiments, the side chain of X_{L} comprises a reactive group. In some embodiments, the side chain of X_{L} comprises a nucleophile. In some embodiments, the side chain of X_{L} comprises an electrophile. In some embodiments, the side chain of X_{L} comprises an amine or a sulfhydryl. In some embodiments, X_{L} is lysine or azido-acetyl lysine. In some embodiments, X_{L} is cysteine. In some embodiments, n is 0. In some embodiments, n is 4. In some embodiments, (X_{S})₄ is Ala-Gly-Ser-Ile. In some embodiments, each amino acid of (X_{S})₄ is independently selected from an aliphatic amino acid, lysine, and serine. In some embodiments, at least one amino acid of (X_{S})₄ is not lysine. In some embodiments, at least one amino acid of (X_{S})₄ is not arginine. In some embodiments, (X_{S})₄ does not comprise a lysine. In some embodiments, (X_{S})₄ does not comprise an arginine. In some embodiments, each of X₁, X₂, and X₃ is selected from alanine, proline, glycine, cysteine, methionine, or tyrosine. In some embodiments, X₁ is alanine or proline. In some embodiments, X₁ is alanine. In some embodiments, X₁ is proline. In some embodiments, X₂ is proline, alanine, or glycine. In some embodiments, X₂ is proline. In some embodiments, X₂ is alanine. In some embodiments, X₂ is glycine. In some embodiments, X₃ is proline, serine, or methionine. In some embodiments, X₃ is proline. In some embodiments, X₃ is serine. In some embodiments, X₃ is methionine. In some embodiments, X₄ is an aliphatic amino acid, serine, or cysteine. In some embodiments, X₄ is a compact amino acid. In some embodiments, X₄ is selected from alanine, serine, glycine, or cysteine. In some embodiments, X₄ is alanine. In some embodiments, X₄ is serine. In some embodiments, X₅ is glycine. In some embodiments, X₆ is a compact amino acid. In some embodiments, X₆ is selected from alanine, proline, serine, glycine, cysteine, methionine, or tyrosine. In some embodiments, X₆ is serine. In some embodiments, X₆ is proline. In some embodiments, X₆ is alanine. In some embodiments, X₇ is a compact amino acid. In some embodiments, X₇ is serine. In some embodiments, X₈ is a compact amino acid. In some embodiments, X₈ is selected from alanine, proline, glycine, cysteine, methionine, or tyrosine. In some embodiments, X₈ is proline. In some embodiments, X₈ is alanine. In some embodiments, X₉ is a compact amino acid. In some embodiments, X₉ is glycine. In some embodiments, the peptide extender comprises at least two cyclic amino acids. In some embodiments, the peptide extender comprises at least three cyclic amino acids. In some embodiments, the peptide extender comprises at least two prolines. In some embodiments, the peptide extender comprises at least three prolines. In some embodiments, the peptide extender comprises exactly three prolines. In some embodiments, the peptide extender comprises at least five, at least six, at least seven, at least eight or at least nine compact amino acids. In some embodiments, each of X₄, X₅, X₆, X₇, X₈, and X₉ are compact amino acids. In some embodiments, at least five of X₄, X₅, X₆, X₇, X₈, and X₉ are compact amino acids. In some embodiments, the peptide extender comprises at least two or at least three glycines. In some embodiments, the peptide extender does not comprise more than two or more than three glycines. In some embodiments, the peptide extender comprises at least two serines. In some embodiments, the peptide extender comprises not more than two or more than three serines. In some embodiments, the peptide extender does not comprise the sequence GSSG. In some embodiments, the peptide extender does not comprise the sequence GSSS. In some embodiments, the peptide extender does not comprise the sequence SSYG. In some embodiments, the peptide extender comprises at least three different amino acid types selected from acidic, basic, aliphatic, cyclic, or aromatic. In some embodiments, the peptide extender comprises at least four different amino acid types selected from acidic, basic, aliphatic, cyclic, or aromatic. In some embodiments, the peptide extender comprises at least five different amino acid types selected from acidic, basic, aliphatic, cyclic, or aromatic. In some embodiments, the peptide extender does not have a motif of ABAB, wherein A is an amino acid of one type and B is an amino acid of another type. In some embodiments, the peptide extender does not have more than three consecutive amino acids of the same type. In some embodiments, X₈ and X₉ are glycine. In some embodiments, X₈ and X₉ are glycine and X₇ is serine. In some embodiments, X₉ is linked to another amino acid by a peptide bond.

### Conjugate Linkers

In certain embodiments, a conjugate linker links a AGTR1 binding conjugate moiety to an oligonucleotide. In certain compounds, a AGTR1 binding conjugate moiety is attached to an oligonucleotide via a conjugate linker through a single bond. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, a compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances compounds comprising a particular conjugate moiety are better taken up by a particular cell type, but once the compound has been taken up, it is desirable that the conjugate group be cleaved to release the unconjugated or parent oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

1. Certain Hexylamino Linkers

In certain embodiments, a compound comprises an oligonucleotide linked to a AGTR1 binding conjugate moiety by a conjugate linker selected from the following structures:

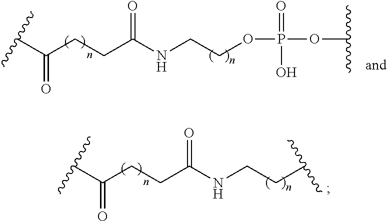

wherein each n is independently selected from 0, 1, 2, 3, 4, 5, 6, or 7.

In certain embodiments, a compound comprises an oligonucleotide linked to a AGTR1 binding conjugate moiety by a conjugate linker selected from the following structures:

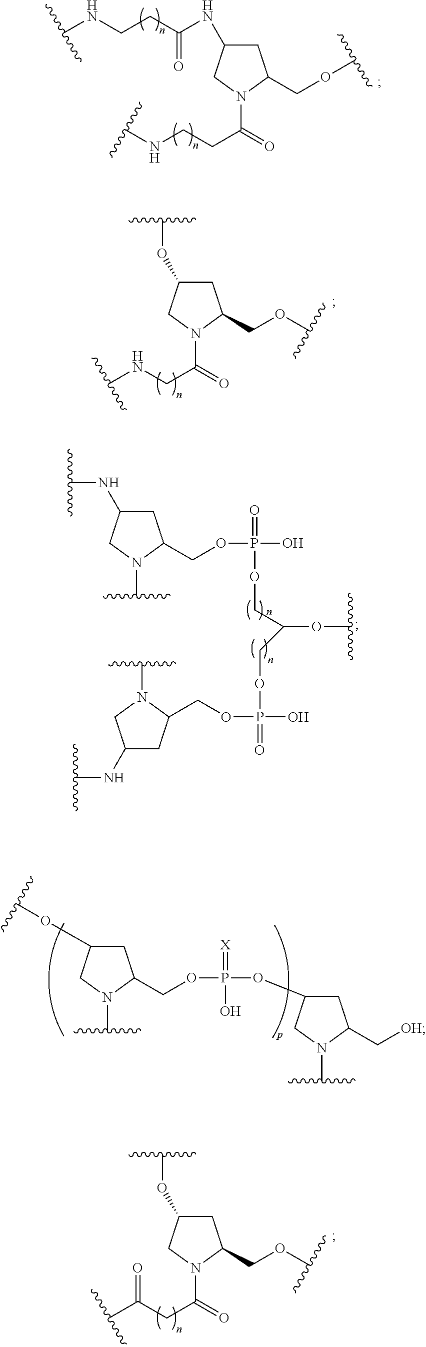

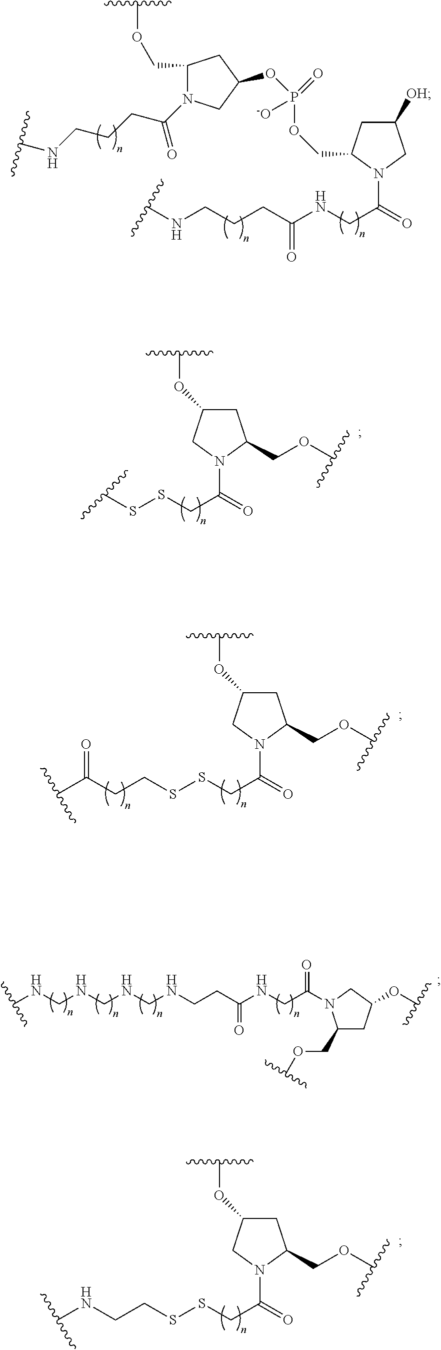

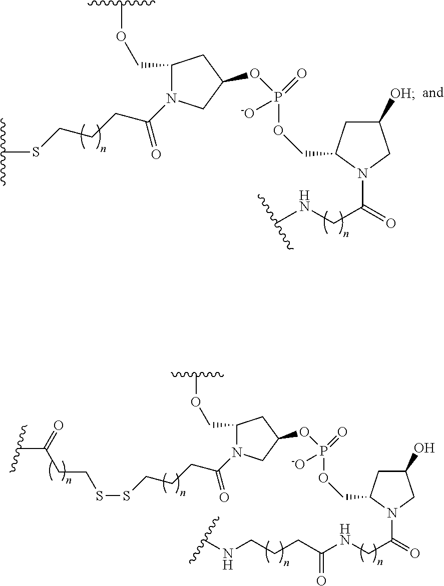

wherein each n is, independently from 1 to 20; and p is from 1 to 6.

In certain embodiments, a compound comprises an oligonucleotide linked to a AGTR1 binding conjugate moiety by a conjugate linker selected from the following structures:

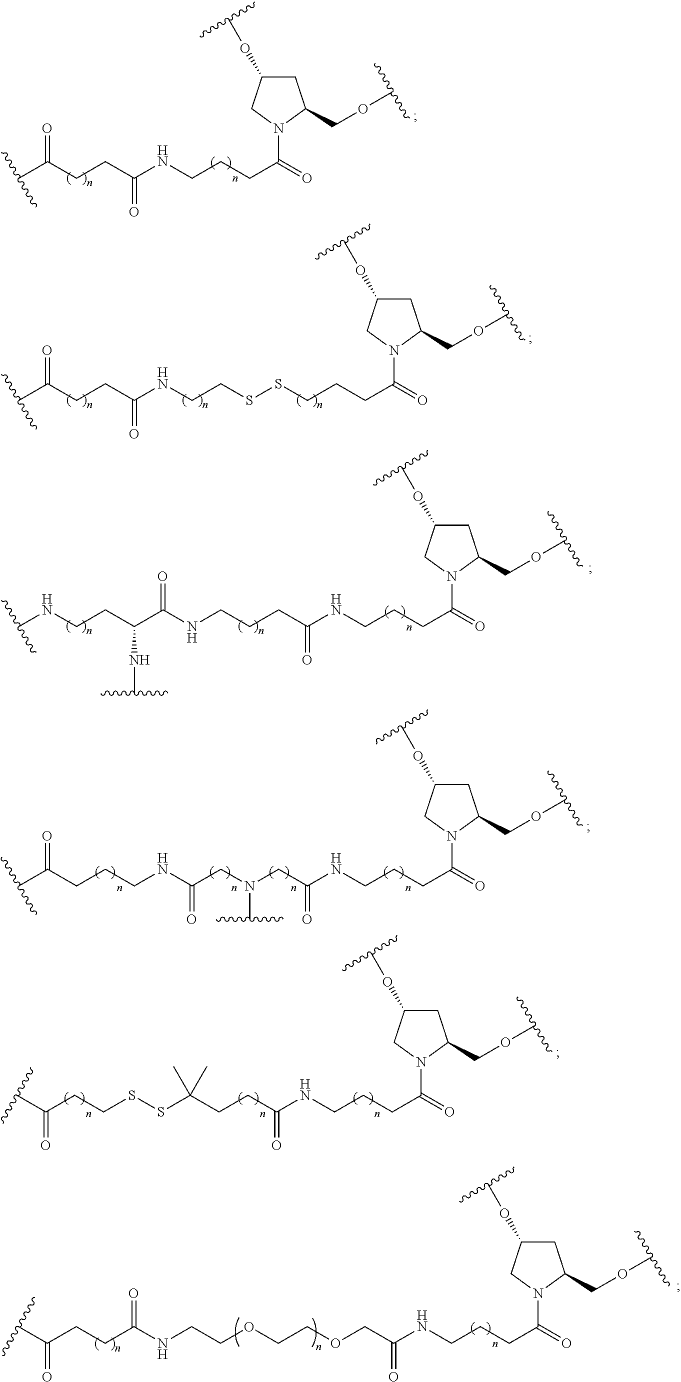

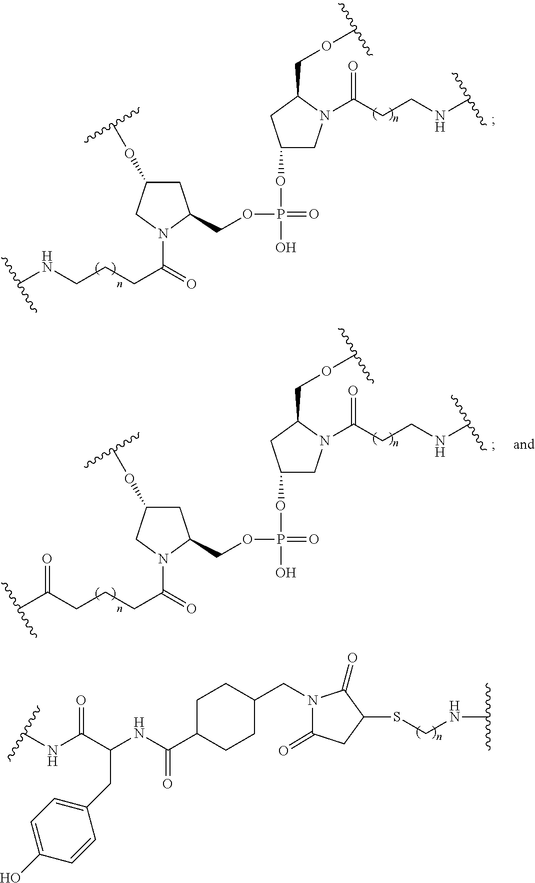

wherein each n is, independently, from 1 to 20.

In certain embodiments, a compound comprises an oligonucleotide linked to a AGTR1 binding conjugate moiety by a conjugate linker selected from the following structures:

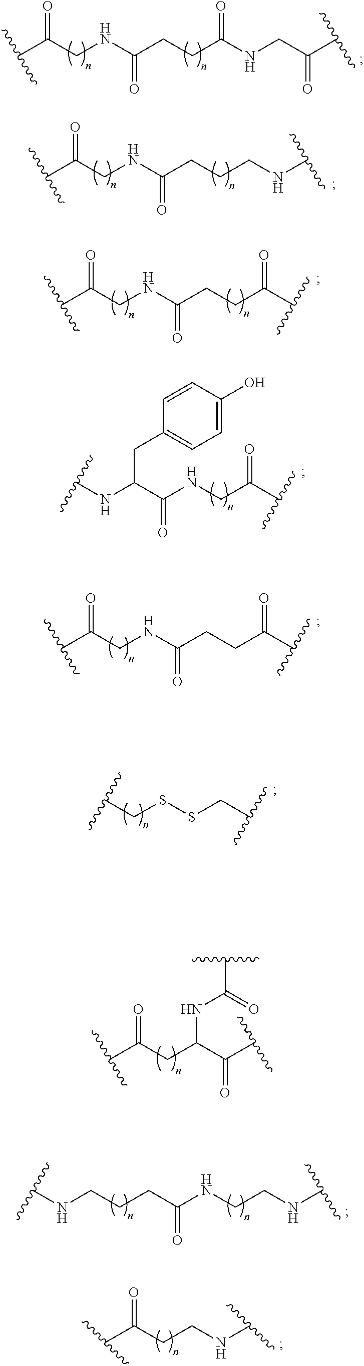

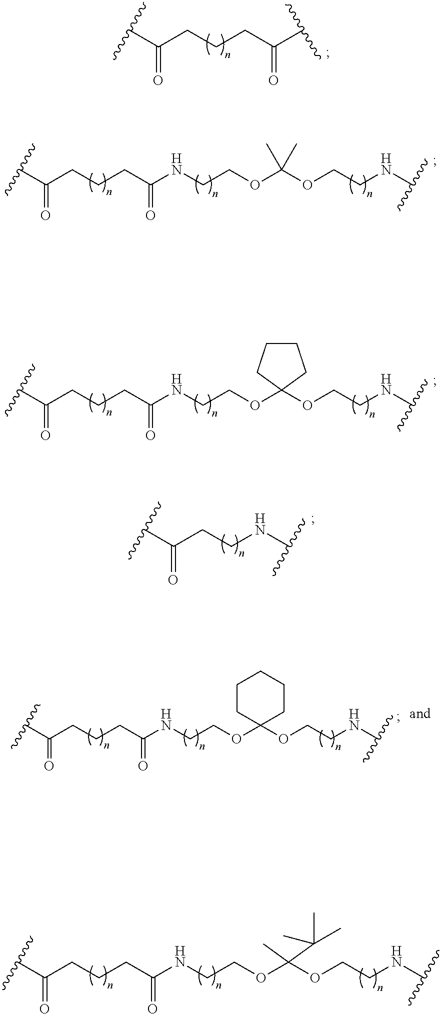

wherein each n is, independently, from 1 to 20.

In certain embodiments, a compound comprises an oligonucleotide linked to a AGTR1 binding conjugate moiety by a conjugate linker selected from the following structures:

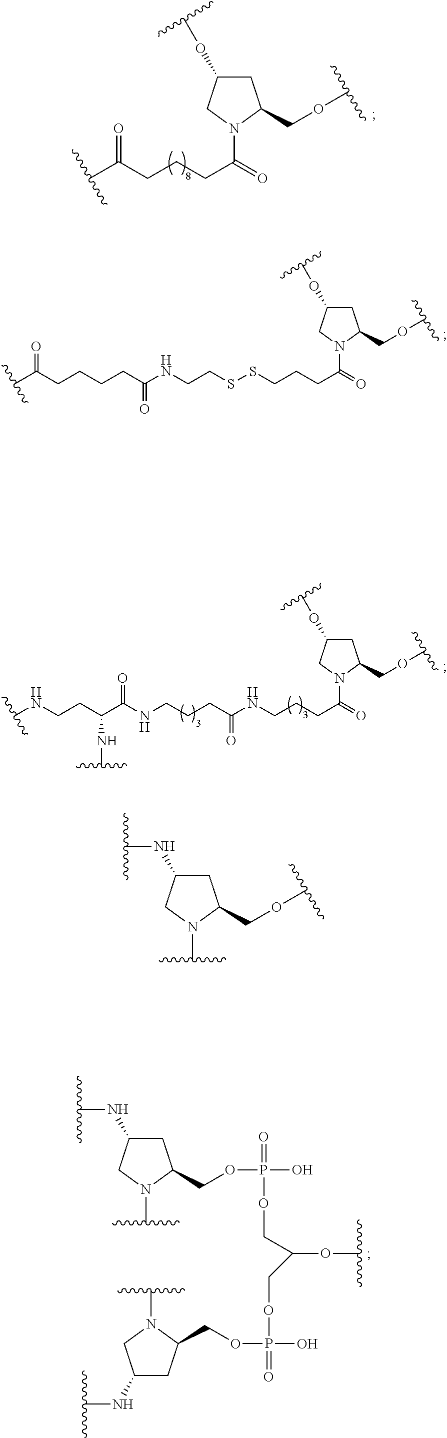

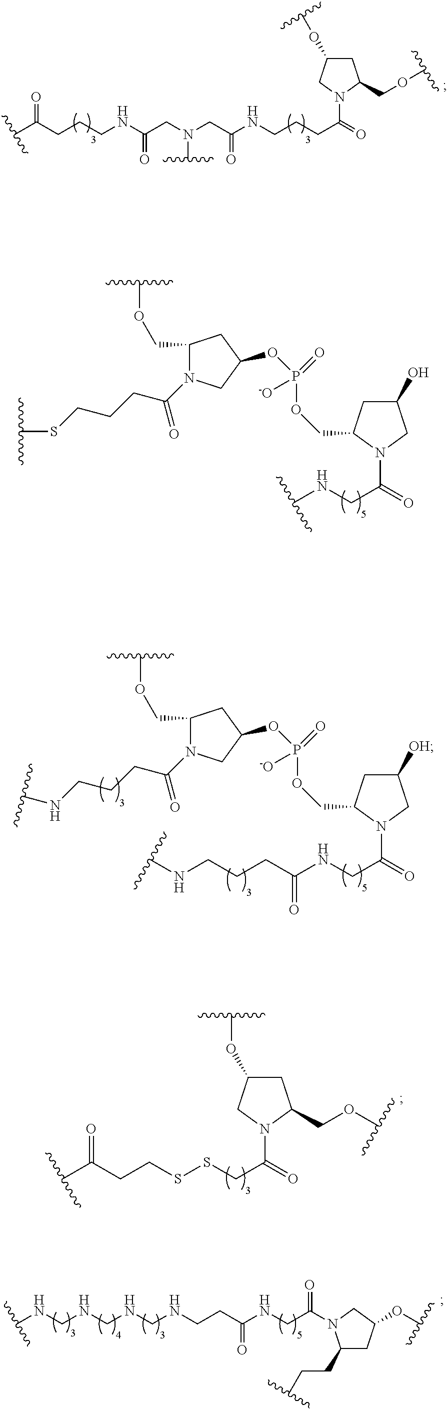

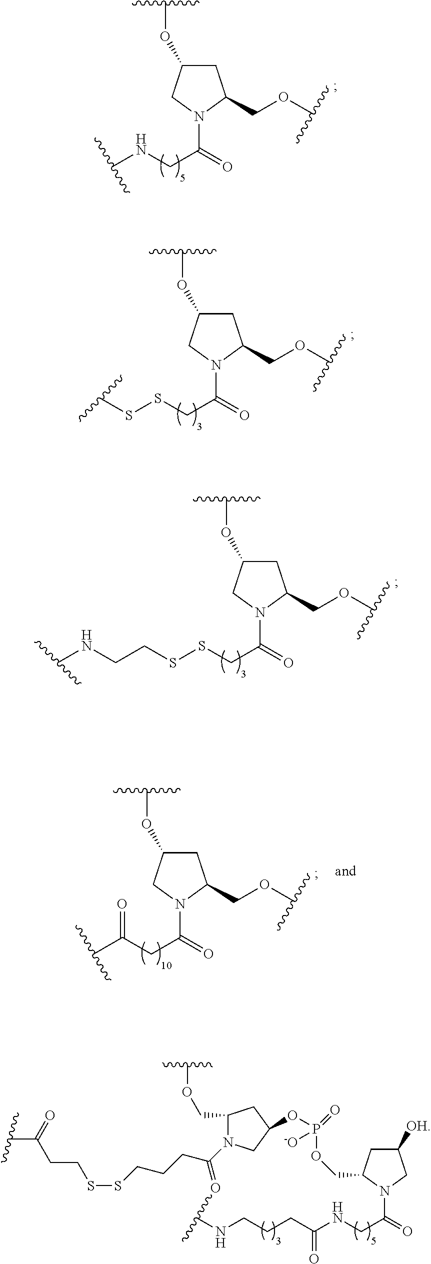

In certain embodiments, a compound comprises an oligonucleotide linked to a AGTR1 binding conjugate moiety by a conjugate linker selected from the following structures:

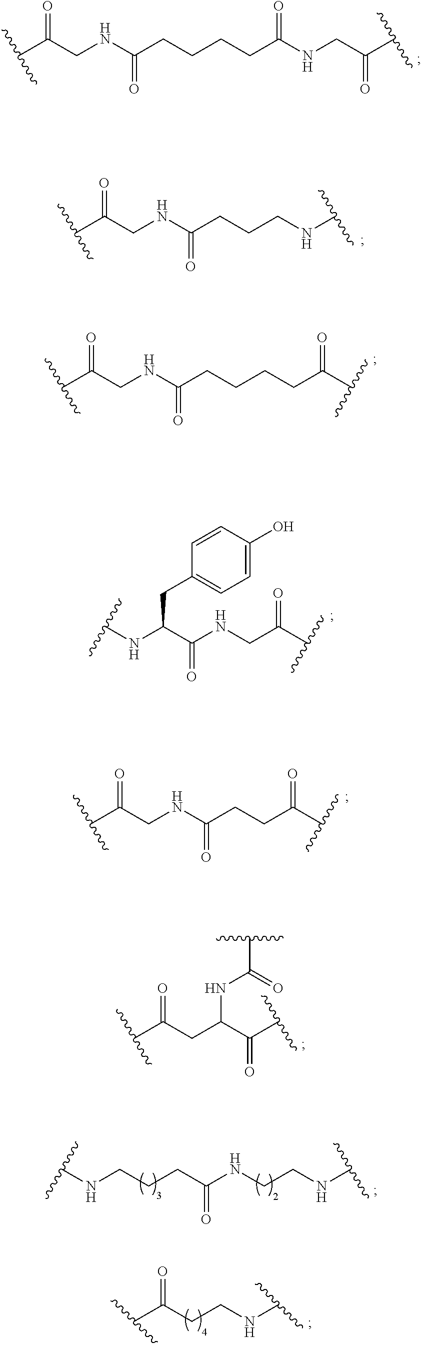

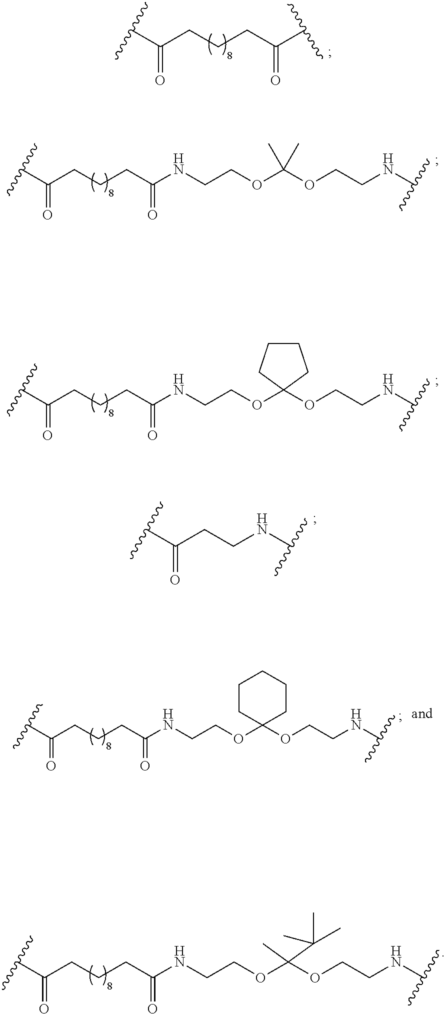

In certain embodiments, a compound comprises an oligonucleotide linked to a AGTR1 binding conjugate moiety by a conjugate linker selected from the following structures:

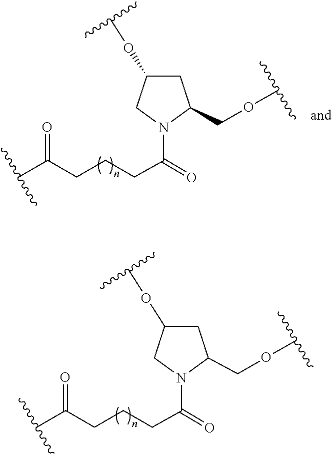

In certain embodiments, a compound comprises an oligonucleotide linked to a AGTR1 binding conjugate moiety by a conjugate linker having the following structure:

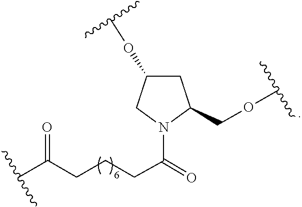

In certain embodiments, a compound comprises an oligonucleotide linked to a AGTR1 binding conjugate moiety by a conjugate linker having the following structure:

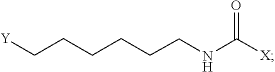

wherein
X directly or indirectly attaches to the AGTR1 binding conjugate moiety; and
Y directly or indirectly attaches to the modified oligonucleotide.

In certain embodiments, a compound comprises an oligonucleotide linked to a AGTR1 binding conjugate moiety by any conjugate linker described in WO 2014/179620, which is incorporated by reference herein in its entirety.

2. Certain Alkyl Phosphate Linkers

In certain embodiments, a compound comprises an oligonucleotide linked to a AGTR1 binding conjugate moiety b a conjugate linker having the following structure:

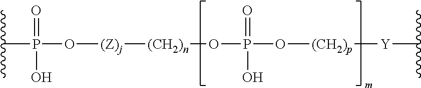

wherein:
the phosphate group is connected to the modified oligonucleotide and Y is connected to the conjugate group;

Y is a phosphodiester or amino (—NH—) group;

Z is a pyrrolidinyl group having the formula:

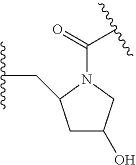 [SMILES: *CC1CC(O)CN1C(*)=O]

j is 0 or 1;

n is from about 1 to about 10;

p is from 1 to about 10;

m is 0 or from 1 to 4; and

when Y is amino then m is 1.

In certain embodiments, Y is amino (—NH—). In certain embodiments, Y is a phosphodiester group. In certain embodiments, n is 3 and p is 3. In certain embodiments, n is 6 and p is 6. In certain embodiments, n is from 2 to 10 and p is from 2 to 10. In certain embodiments, n and p are different. In certain embodiments, n and p are the same. In certain embodiments, m is 0. In certain embodiments, m is 1. In certain embodiments, j is 0. In certain embodiments, j is 1 and Z has the formula:

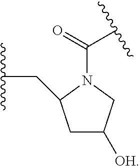 [SMILES: *CC1CC(O)CN1C(*)=O]

In certain embodiments, wherein n is 2 and p is 3. In certain embodiments, n is 5 and p is 6.

In certain embodiments, a compound comprises an oligonucleotide linked to a AGTR1 binding conjugate moiety by a conjugate linker having the following structure:

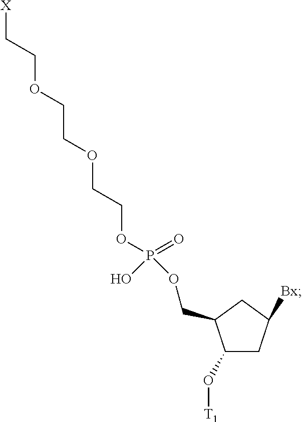

wherein X directly or indirectly attaches to the AGTR1 binding conjugate moiety; and
wherein T₁ comprises the modified oligonucleotide; and Bx is a modified or unmodified nucleobase.

3. Certain Click Chemistry Linkers

In certain embodiments, a compound comprises an oligonucleotide linked to a AGTR1 binding conjugate moiety by a conjugate linker, wherein the conjugate linker is prepared using Click chemistry known in the art. Compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J. Am. Chem. Soc. 2012, 134(28), 11618-11631), which is incorporated by reference herein in its entirety. Additional linkers suitable for use in several embodiments can be prepared by Click chemistry described in “Click Chemistry for Biotechnology and Materials Science” Ed. Joerg Laham, Wiley 2009, which is incorporated by reference herein in its entirety.

In certain embodiments, a Click reaction can be used to link a AGTR1 binding conjugate moiety and an oligonucleotide by reacting:

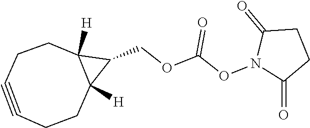 [SMILES: O=C(OC[C@@H]1[C@@H]2CCC#CCC[C@@H]21)ON1C(=O)CCC1=O]

with an oligonucleotide having a terminal amine, including but not limited to the following compound:

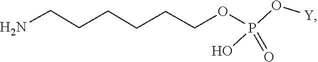

wherein Y is directly or indirectly attached to the oligonucleotide or is the remainder of the oligonucleotide, to yield:

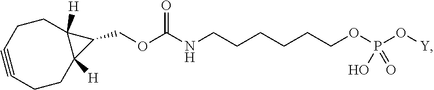

which can be reacted with a AGTR1 binding conjugate moiety having an azide to yield:

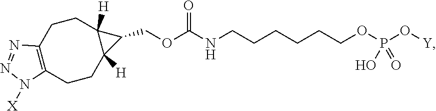

wherein N—N═N is formed from an azido group of the AGTR1 binding conjugate moiety and X is the remainder of the AGTR1 binding conjugate moiety.

In certain embodiments, a compound comprises an oligonucleotide linked to a AGTR1 binding conjugate moiety by a conjugate linker, wherein the conjugate linker is prepared from the following compound:

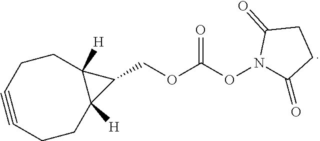 [SMILES: O=C(OC[C@@H]1[C@@H]2CCC#CCC[C@@H]21)ON1C(=O)CCC1=O]

In certain embodiments, a compound comprises an oligonucleotide linked to a AGTR1 binding conjugate moiety by a conjugate linker, wherein the conjugate linker comprises:

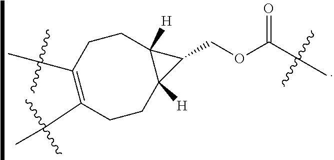 [SMILES: *C(=O)OC[C@@H]1[C@@H]2CC/C(*)=C(/*)CC[C@@H]21]

In certain embodiments, a compound comprises an oligonucleotide linked to a AGTR1 binding conjugate moiety by a conjugate linker, wherein the conjugate linker comprises:

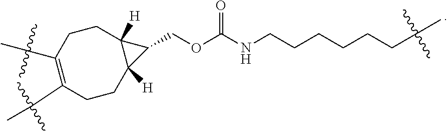 [SMILES: *CCCCCCNC(=O)OC[C@@H]1[C@@H]2CC/C(*)=C(/*)CC[C@@H]21]

In certain embodiments, a compound comprises an oligonucleotide linked to a AGTR1 binding conjugate moiety by a conjugate linker, wherein the compound comprises:

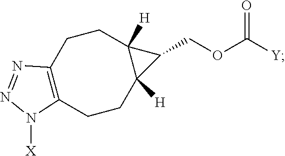

wherein N—N═N is formed from an azido group of the AGTR1 binding conjugate moiety and X is the remainder of the AGTR1 binding conjugate moiety; and Y directly or indirectly attaches to the oligonucleotide.

In certain embodiments, a compound comprises an oligonucleotide linked to a AGTR1 binding conjugate moiety by a conjugate linker, wherein the compound comprises:

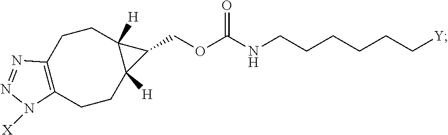

wherein N—N═N is formed from an azido group of the AGTR1 binding conjugate moiety and X is the remainder of the AGTR1 binding conjugate moiety; and Y directly or indirectly attaches to the oligonucleotide.

In certain embodiments, a compound comprises an oligonucleotide linked to a AGTR1 binding conjugate moiety by a conjugate linker, wherein the compound comprises:

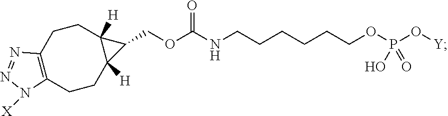

wherein N—N═N represents an azido group of the AGTR1 binding conjugate moiety and X directly or indirectly attaches to the remainder of the AGTR1 binding conjugate moiety; and Y directly or indirectly attaches to the oligonucleotide, or is the remainder of the oligonucleotide

In certain embodiments, a compound comprises an oligonucleotide linked to a AGTR1 binding conjugate moiety by a conjugate linker, wherein the conjugate linker is prepared using Click chemistry and disulfide linkages.

In certain embodiments, a compound comprises an oligonucleotide linked to a AGTR1 binding conjugate moiety by a conjugate linker, wherein the compound comprises:

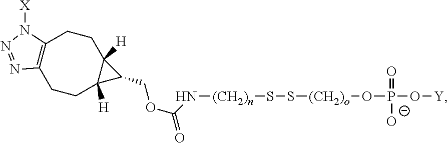

wherein N—N═N is formed from an azido group of the AGTR1 binding conjugate moiety and X is the remainder of the AGTR1 binding conjugate moiety; n and o are independently selected from 2 to 10; and Y directly or indirectly attaches to the oligonucleotide, or is the remainder of the oligonucleotide.

In certain embodiments, a compound comprises an oligonucleotide linked to a AGTR1 binding conjugate moiety by a conjugate linker, wherein the compound comprises:

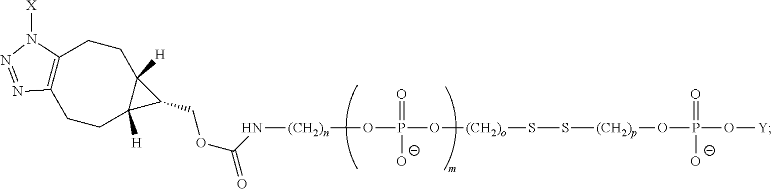

wherein N—N═N is formed from an azido group of the AGTR1 binding conjugate moiety and X directly or indirectly attaches to the remainder of the AGTR1 binding conjugate moiety; n, o, and p are independently selected from 2 to 10; m is 0 or 1; and Y directly or indirectly attaches to the oligonucleotide, or is the remainder of the oligonucleotide.

In certain embodiments, a compound comprises an oligonucleotide linked to a AGTR1 binding conjugate moiety by a conjugate linker, wherein the compound comprises:

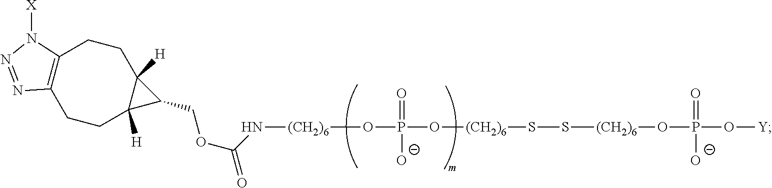

wherein N—N═N is formed from an azido group of the AGTR1 binding conjugate moiety and X directly or indirectly attaches to the remainder of the AGTR1 binding conjugate moiety; m is 0 or 1; and Y directly or indirectly attaches to the oligonucleotide, or is the remainder of the oligonucleotide.

In certain embodiments, a compound comprises an oligonucleotide linked to a AGTR1 binding conjugate moiety by a conjugate linker, wherein the compound comprises:

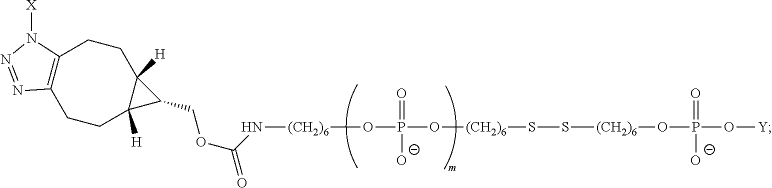

wherein N—N═N is formed from an azido group of the AGTR1 binding conjugate moiety and X directly or indirectly attaches to the remainder of the AGTR1 binding conjugate moiety; m is 1; and Y directly or indirectly attaches to the oligonucleotide, or is the remainder of the oligonucleotide.

In certain embodiments, a compound comprises an oligonucleotide linked to a AGTR1 binding conjugate moiety by a conjugate linker, wherein the compound comprises:

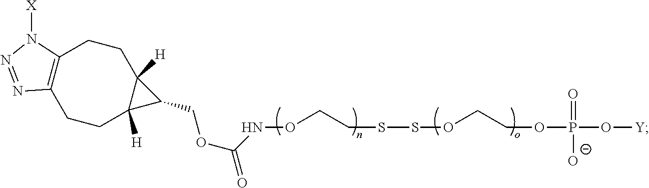

wherein N—N═N is formed from an azido group of the AGTR1 binding conjugate moiety and X directly or indirectly attaches to the remainder of the AGTR1 binding conjugate moiety; n and o are independently selected from 2 to 10; and Y directly or indirectly attaches to the oligonucleotide, or is the remainder of the oligonucleotide.

4. Certain Maleimide and Maleimide Acid Linkers

In certain embodiments, a compound comprises an oligonucleotide linked to a AGTR1 binding conjugate moiety by a conjugate linker, wherein the conjugate linker comprises:

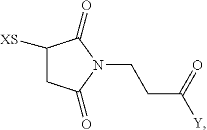

wherein X directly or indirectly attaches to the AGTR1 binding conjugate moiety; and Y directly or indirectly attaches to the oligonucleotide.

In certain embodiments, the above conjugate linker can link a peptide to an oligonucleotide. In certain embodiments, a compound comprises an oligonucleotide linked to a peptide by a conjugate linker, wherein the conjugate linker comprises:

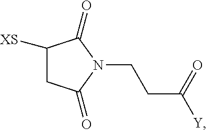

wherein X directly or indirectly attaches to the peptide; and Y directly or indirectly attaches to the oligonucleotide.

In certain embodiments, a compound comprises an oligonucleotide linked to a AGTR1 binding conjugate moiety, such as a peptide, by a conjugate linker, wherein the conjugate linker comprises:

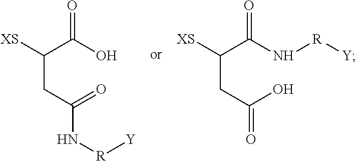

wherein R═(CH₂)ₙ and n is from 1 to 12; X directly or indirectly attaches to the AGTR1 binding conjugate moiety, such as a peptide; and Y directly or indirectly attaches to the oligonucleotide.

In certain embodiments, a compound comprises an oligonucleotide linked to a AGTR1 binding conjugate moiety, such as a peptide, by a conjugate linker, wherein the conjugate linker comprises:

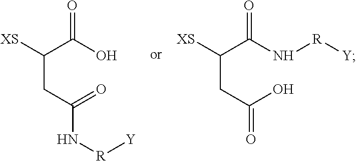

wherein

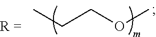

m is from 1 to 12; X directly or indirectly attaches to the AGTR1 binding conjugate moiety, such as a peptide; and Y directly or indirectly attaches to the oligonucleotide.

In certain embodiments, a composition comprises or consists of a substantially pure mixture of two compounds, wherein the first compound comprises an oligonucleotide linked to a AGTR1 binding conjugate moiety, such as a peptide, by a conjugate linker, wherein the conjugate linker comprises:

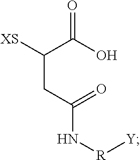

wherein R═(CH₂)ₙ and n is from 1 to 12; X directly or indirectly attaches to the AGTR1 binding conjugate moiety, such as a peptide; and Y directly or indirectly attaches to the oligonucleotide; and the second compound comprises an oligonucleotide linked to a AGTR1 binding conjugate moiety, such as a peptide, by a conjugate linker, wherein the conjugate linker comprises:

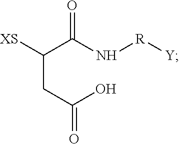

wherein R═(CH₂)ₙ and n is from 1 to 12; X directly or indirectly attaches to the AGTR1 binding conjugate moiety, such as a peptide; and Y directly or indirectly attaches to the oligonucleotide.

In certain embodiments, a composition comprises or consists of a substantially pure mixture of two compounds, wherein the first compound comprises an oligonucleotide linked to a AGTR1 binding conjugate moiety, such as a peptide, by a conjugate linker, wherein the conjugate linker comprises:

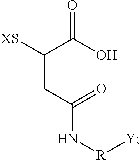

wherein

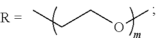

m is from 1 to 12; X directly or indirectly attaches to the AGTR1 binding conjugate moiety, such as a peptide; and Y directly or indirectly attaches to the oligonucleotide; and the second compound comprises an oligonucleotide linked to a AGTR1 binding conjugate moiety, such as a peptide, b a conjugate linker, wherein the conjugate linker comprises:

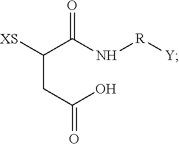

wherein

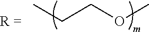

and m is from 1 to 12; X directly or indirectly attaches to the AGTR1 binding conjugate moiety, such as a peptide; and Y directly or indirectly attaches to the oligonucleotide.

In certain embodiments, a compound comprises an oligonucleotide linked to a AGTR1 binding conjugate moiety, such as a peptide, by a conjugate linker, wherein the conjugate linker comprises:

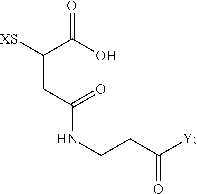

wherein X directly or indirectly attaches to the AGTR1 binding conjugate moiety, such as a peptide; and Y directly or indirectly attaches to the oligonucleotide.

In certain embodiments, a compound comprises an oligonucleotide linked to a AGTR1 binding conjugate moiety, such as a peptide, by a conjugate linker, wherein the conjugate linker comprises:

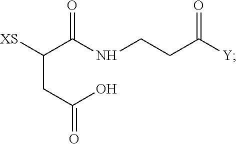

wherein X directly or indirectly attaches to the AGTR1 binding conjugate moiety, such as a peptide; and Y directly or indirectly attaches to the oligonucleotide.

In certain embodiments, a composition comprises or consists of a substantially pure mixture of two compounds, wherein the first compound comprises an oligonucleotide linked to a AGTR1 binding conjugate moiety, such as a peptide, by a conjugate linker, wherein the conjugate linker comprises:

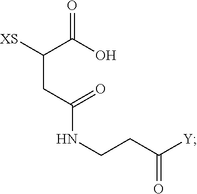

wherein X directly or indirectly attaches to the AGTR1 binding conjugate moiety, such as a peptide; and Y directly or indirectly attaches to the oligonucleotide;
and the second compound comprises an oligonucleotide linked to a AGTR1 binding conjugate moiety, such as a peptide, by a conjugate linker, wherein the conjugate linker comprises:

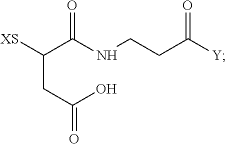

wherein X directly or indirectly attaches to the AGTR1 binding conjugate moiety, such as a peptide; and Y directly or indirectly attaches to the oligonucleotide.

5. Certain Disulfide Linkages

In certain embodiments, a compound comprises an oligonucleotide linked to a AGTR1 binding conjugate moiety by a conjugate linker, wherein the conjugate linker comprises a disulfide linkage. In certain embodiments, oligonucleotides comprise activated disulfides which form a disulfide linkage with a AGTR1 binding conjugate moiety. In certain embodiments, a compound comprises an oligonucleotide comprising an activated disulfide moiety capable of forming a cleavable or reversible bond with a AGTR1 binding conjugate moiety. In certain embodiments, a compound comprises an oligonucleotide directly attached to a AGTR1 binding conjugate moiety by a disulfide bond without a conjugate linker.

In certain embodiments, a compound comprises a linker between an oligonucleotide and activated disulfide moiety. In another embodiment, the activated disulfide moiety has the formula —S—S(O)2-substituted or unsubstituted C1-C12 alkyl or —S—S—C(O)O-substituted or unsubstituted C1-C12 alkyl. Preferred activated disulfide moieties are methane thiosulfonate and dithiocarbomethoxy. In further embodiments, the activated disulfide is substituted or unsubstituted dithiopyridyl, substituted or unsubstituted dithiobenzothiazolyl, or substituted or unsubstituted dithiotetrazolyl. Preferred activated disulfides are 2-dithiopyridyl, 2-dithio-3-nitropyridyl, 2-dithio-5-nitropyridyl, 2-dithiobenzothiazolyl, N—(C1-C12 alkyl)-2-dithiopyridyl, 2-dithiopyridyl-N-oxide, or 2-dithio-1-methyl-1H-tetrazolyl.

In some embodiments, the activated disulfide moiety has the formula —S—S(O)ₙ—R₁, wherein
n is 0, 1, or 2; and
R₁ is selected from substituted or unsubstituted heterocyclic, substituted or unsubstituted aliphatic, or —C(O)O—R₂, wherein R₂ is substituted or unsubstituted aliphatic.

In another embodiment, the activated disulfide moiety has the formula —S—S(O)₂-substituted or unsubstituted C₁-C₁₂ alkyl or —S—S—C(O)O-substituted or unsubstituted C₁-C₁₂ alkyl. In certain embodiments, activated disulfide moieties include methane thiosulfonate and dithiocarbomethoxy. In further embodiments, the activated disulfide can be substituted or unsubstituted dithiopyridyl, substituted or unsubstituted dithiobenzothiazolyl, or substituted or unsubstituted dithiotetrazolyl. Further examples of activated disulfides include but are not limited to 2-dithiopyridyl, 2-dithio-3-nitropyridyl, 2-dithio-5-nitropyridyl, 2-dithiobenzothiazolyl, N—(C₁-C₁₂ alkyl)-2-dithiopyridyl, 2-dithiopyridyl-N-oxide, and 2-dithio-1-methyl-1H-tetrazolyl.

In some embodiments, the bivalent linking group is a bivalent substituted or unsubstituted aliphatic group. In another embodiment, the bivalent linking group has the formula -Q₁-G-Q₂-, wherein

Q₁ and Q₂ are independently absent or selected from substituted or unsubstituted C₁-C₁₂ alkylene, substituted or unsubstituted alkarylene or —(CH₂)ₘ—O—(CH₂)ₚ—, wherein

each m and p are, independently, an integer from 1 to about 10;

G is —NH—C(O)—, —C(O)—NH—, —NH—C(O)—NH—, —NH—C(S)—NH—, —NH—O—, NH—C(O)—O—, or —O—CH₂—C(O)—NH—.

Examples of bivalent linking groups include but are not limited to:

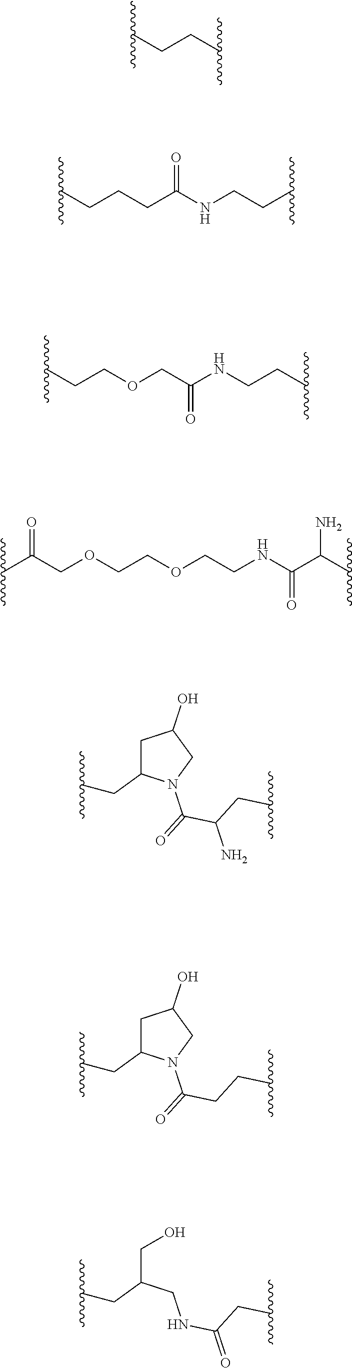

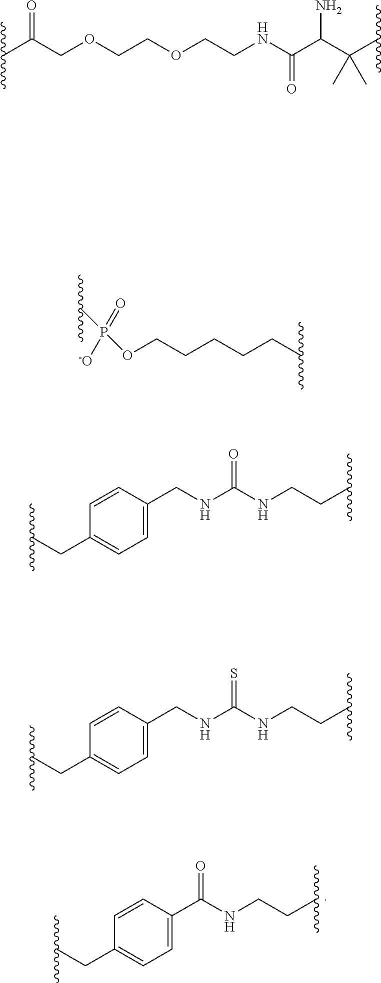

In certain embodiments, a compound comprises an oligonucleotide linked to a AGTR1 binding conjugate moiety by a disulfide linkage described in U.S. Pat. No. 7,713,944, which is incorporated by reference herein in its entirety. In certain embodiments, a compound comprises an oligonucleotide linked to a AGTR1 binding conjugate moiety wherein the oligonucleotide comprises an activated disulfide described in U.S. Pat. No. 7,713,944, which is incorporated by reference herein in its entirety.

In certain embodiments, any of the above compounds comprising an oligonucleotide linked to a AGTR1 binding conjugate moiety by a disulfide linkage, whether directly or by a conjugate linker described herein, can comprise a disulfide linkage between a cysteine, penicillamine, homocysteine, mercaptopropionic acid, or β-Mercapto-β,β,-cyclopentamethylene propionic acid moiety of the AGTR1 binding conjugate moiety and the oligonucleotide or conjugate linker. In certain embodiments, a compound comprises an oligonucleotide directly linked to a AGTR1 binding conjugate moiety by a disulfide linkage. In certain embodiments a compound comprises an oligonucleotide directly linked to a AGTR1 binding conjugate moiety by a disulfide linkage, wherein the disulfide linkage is between the oligonucleotide and a cysteine, penicillamine, homocysteine, mercaptopropionic acid, or β-Mercapto-β,β,-cyclopentamethylene propionic acid moiety of the AGTR1 binding conjugate moiety. In certain embodiments, a compound comprises an oligonucleotide, conjugate linker, and AGTR1 binding conjugate moiety wherein a disulfide linkage links the conjugate linker and the AGTR1 binding conjugate moiety, and the oligonucleotide is attached to the conjugate linker. In certain embodiments, a compound comprises an oligonucleotide, conjugate linker, and AGTR1 binding conjugate moiety wherein a disulfide linkage links the conjugate linker to a cysteine, penicillamine, homocysteine, mercaptopropionic acid, or β-Mercapto-β,β,-cyclopentamethylene propionic acid moiety of the AGTR1 binding conjugate moiety, and the oligonucleotide is attached to the conjugate linker. In certain embodiments, the cysteine, penicillamine, homocysteine, mercaptopropionic acid, or 0-Mercapto-β,β,-cyclopentamethylene propionic acid moiety is at the N-terminus, C-terminus, side chain, or internal amino acid position of the AGTR1 binding conjugate moiety.

6 Certain Enzyme Cleavable Linkages

In certain embodiments, a compound comprises an oligonucleotide linked to a AGTR1 binding conjugate moiety by a conjugate linker, wherein the conjugate linker comprises an enzyme cleavable moiety. In certain embodiments, the AGTR1 binding conjugate moiety is a AGTR1 binding conjugate moiety. In certain embodiments, the enzyme cleavable moiety is a peptide, such as a dipeptide.

Enzymes known in the art for use in activating prodrugs can be used to cleave an enzyme cleavable moiety provided in certain embodiments. In certain embodiments, an enzyme cleavable moiety can be cleaved by DT diaphorase, plasmin, carboxypeptidase G2, thymidine kinase (viral), cytosine deaminase, glucose oxidase, xanthine oxidase, carboxypeptidase A, α-galactosidase, β-glucosidase, azoreductase, γ-glutamyltransferase, β-glucuronidase, β-lactamase, alkaline phosphatase, aminopeptidase, penicillin amidase or nitroreductase.

In certain embodiments, the enzyme cleavable moiety is cleavable by a protease or peptidase. In certain embodiments, the enzyme cleavable moiety is cleavable by a protease or peptidase selected from: gastricsin, memapsin-2, chymosin, renin, renin-2, cathepsin D, cathepsin E, penicillopepsin, rhizopuspepsin, mucorpepsin, barrierpepsin, aspergillopepsin I, endothiapepsin, saccharopepsin, phytepsin, plasmepsin-1, plasmepsin-2, yapsin-1, yapsin-2, nepenthesin, memapsin-1, napsin A, HIV-1 retropepsin, HIV-2 retropepsin, simian immunodeficiency virus retropepsin, equine infectious anaemia virus retropepsin, feline immunodeficiency virus retropepsin, murine leukemia virus-type retropepsin, Mason-Pfizer leukemia virus retropepsin, human endogenous retrovirus K retropepsin, retropepsin (human T-cell leukemia virus), bovine leukemia virus retropepsin, Rous sarcoma virus retropepsin, scytalidoglutamic peptidase, aspergilloglutamic peptidase, thermopsin, signal peptidase II, spumapepsin, type 4 prepilin peptidase 1, omptin, plasminogen activator Pla, papain, chymopapain, caricain, glycyl endopeptidase, stem bromelain, ficain, actinidain, cathepsin V, vignain, cathepsin X, zingipain, cathepsin F, ananain, fruit bromelain, cathepsin L, cathepsin L1 (Fasciola sp.), cathepsin S, cathepsin K, cathepsin H, aleurain, histolysain, cathepsin B, dipeptidyl-peptidase I, peptidase 1 (mite), CPB peptidase, cruzipain, V-cath peptidase, bleomycin hydrolase (subject), bleomycin hydrolase (yeast), aminopeptidase C, CPC peptidase, calpain-1, calpain-2, calpain-3, Tpr peptidase (Porphyromonas gingivalis), poliovirus-type picomain 3C, hepatitis A virus-type picomain 3C, human rhinovirus 2-type picornain 3C, foot-and-mouth disease virus picomain 3C, enterovirus picomain 2A, rhinovirus picomain 2A, nuclear-inclusion-a peptidase (plum pox virus), tobacco etch virus NIa peptidase, adenain, potato virus Y-type helper component peptidase, sindbis virus-type nsP2 peptidase, streptopain, clostripain, ubiquitinyl hydrolase-L1, ubiquitinyl hydrolase-L3, legumain (plant beta form), legumain, subject-type, caspase-1, caspase-3, caspase-7, caspase-6, caspase-8, caspase-9, pyroglutamyl-peptidase I (prokaryote), pyroglutamyl-peptidase I (chordate), murine hepatitis coronavirus papain-like peptidase 1, ubiquitin-specific peptidase 5, tymovirus peptidase, rabbit hemorrhagic disease virus 3C-like peptidase, gingipain RgpA, gingipain Kgp, gamma-glutamyl hydrolase, foot-and-mouth disease virus L-peptidase, porcine transmissible gastroenteritis virus-type main peptidase, calicivirin, staphopain A, Ulp1 peptidase, separase (yeast-type), YopJ protein, PfpI peptidase, sortase A (Staphylococcus-type), aminopeptidase N, lysyl aminopeptidase (bacteria), aminopeptidase A, leukotriene A₄ hydrolase, pyroglutamyl-peptidase II, cytosol alanyl aminopeptidase, cystinyl aminopeptidase, aminopeptidase B, aminopeptidase Ey, angiotensin-converting enzyme peptidase unit 1, peptidyl-dipeptidase Acer, angiotensin-converting enzyme peptidase unit 2, angiotensin-converting enzyme-2, thimet oligopeptidase, neurolysin, saccharolysin, oligopeptidase A, peptidyl-dipeptidase Dcp, mitochondrial intermediate peptidase, oligopeptidase F, thermolysin, vibriolysin, pseudolysin, coccolysin, aureolysin, stearolysin, mycolysin, snapalysin, leishmanolysin, bacterial collagenase V, bacterial collagenase G/A, matrix metallopeptidase-1, matrix metallopeptidase-8, matrix metallopeptidase-2, matrix metallopeptidase-9, matrix metallopeptidase-3, matrix metallopeptidase-10 (Homo sapiens-type), matrix metallopeptidase-11, matrix metallopeptidase-7, matrix metallopeptidase-12, envelysin, matrix metallopeptidase-13, membrane-type matrix metallopeptidase-1, membrane-type matrix metallopeptidase-2, matrix metallopeptidase-20, fragilysin, matrix metallopeptidase-26, serralysin, aeruginolysin, gametolysin, astacin, meprin alpha subunit, procollagen C-peptidase, choriolysin L, choriolysin H, fiavastacin, fibrolase, jararhagin, adamalysin, atrolysin A, atrolysin B, atrolysin C, atrolysin E, atroxase, russellysin, ADAM1 peptidase, ADAM9 peptidase, ADAM10 peptidase, Kuzbanian peptidase (non-mammalian), ADAM12 peptidase, ADAM17 peptidase, ADAMTS4 peptidase, ADAMTS1 peptidase, ADAMTS5 peptidase, ADAMTS13 peptidase, procollagen I N-peptidase, neprilysin, endothelin-converting enzyme 1, oligopeptidase O1, neprilysin-2, PHEX peptidase, carboxypeptidase A1, carboxypeptidase A₂, carboxypeptidase B, carboxypeptidase N, carboxypeptidase E, carboxypeptidase M, carboxypeptidase T, carboxypeptidase B2, carboxypeptidase A3, metallocarboxypeptidase D peptidase unit 1, metallocarboxypeptidase D peptidase unit 2, zinc D-Ala-D-Ala carboxypeptidase (Streptomyces-type), vanY D-Ala-D-Ala carboxypeptidase, vanX D-Ala-D-Ala dipeptidase, pitrilysin, insulysin, mitochondrial processing peptidase beta-subunit, nardilysin, leucine aminopeptidase 3, leucyl aminopeptidase (plant-type), aminopeptidase I, aspartyl aminopeptidase, membrane dipeptidase, glutamate carboxypeptidase, peptidase T, carboxypeptidase Ssl, beta-lytic metallopeptidase, staphylolysin, lysostaphin, methionyl aminopeptidase 1 (Escherichia-type), methionyl aminopeptidase 2, Xaa-Pro dipeptidase (bacteria-type), aminopeptidase P (bacteria), aminopeptidase P2, Xaa-Pro dipeptidase (eukaryote), IgA1-specific metallopeptidase, tentoxilysin, bontoxilysin, aminopeptidase Y, aminopeptidase Ap1, aminopeptidase S (Streptomyces-type), glutamate carboxypeptidase II, carboxypeptidase Taq, anthrax lethal factor, deuterolysin, peptidyl-Lys metallopeptidase, FtsH peptidase, m-AAA peptidase, i-AAA peptidase, AtFtsH2 peptidase, pappalysin-1, Ste24 peptidase, dipeptidyl-peptidase III, site 2 peptidase, sporulation factor SpoIVFB, HybD peptidase, gpr peptidase, chymotrypsin A (cattle-type), granzyme B (Homo sapiens-type), factor VII-activating peptidase, trypsin (Streptomyces griseus-type), hypodermin C, elastase-2, cathepsin G, myeloblastin, granzyme A, granzyme M, chymase (Homo sapiens-type), mast cell peptidase 1 (Rattus-type), duodenase, tryptase alpha, granzyme K, mast cell peptidase 5 (mouse numbering), trypsin 1, chymotrypsin B, elastase-1, pancreatic endopeptidase E, pancreatic elastase II, enteropeptidase, chymotrypsin C, prostasin, kallikrein 1, kallikrein-related peptidase 2, kallikrein-related peptidase 3, kallikrein 1 (Mus musculus), kallikrein 1-related peptidase b3, kallikrein 1-related peptidase c2 (Rattus norvegicus), kallikrein 13 (Mus musculus), ancrod, bothrombin, complement factor D, complement component activated C1r, complement component activated C1s, complement factor Bb, mannan-binding lectin-associated serine peptidase 1, complement factor I, coagulation factor XIIa, plasma kallikrein, coagulation factor XIa, coagulation factor IXa, coagulation factor Vila, coagulation factor Xa, thrombin, protein C (activated), coagulation factor C (Limulus, Tachypleus), activated, coagulation factor B (Limulus, Tachypleus), activated, clotting enzyme (Tachypleus-type), acrosin, hepsin, mannan-binding lectin-associated serine peptidase 2, urokinase-type plasminogen activator, t-plasminogen activator, plasmin, kallikrein-related peptidase 6, plasminogen activator (Desmodus-type), kallikrein-related peptidase 8, kallikrein-related peptidase 4, streptogrisin A, streptogrisin B, streptogrisin E, alpha-lytic endopeptidase, glutamyl peptidase I, DegP peptidase, HtrA2 peptidase, lysyl endopeptidase (bacteria), kallikrein-related peptidase 7, matriptase, togavirin, IgA1-specific serine peptidase (Neisseria-type), flavivirin, subtilisin Carlsberg, subtilisin lentus, thermitase, subtilisin Ak1, lactocepin I, C5a peptidase, dentilisin, subtilisin BPN′, subtilisin E, aqualysin 1, cerevisin, oryzin, endopeptidase K, thermomycolin, site-1 peptidase, kexin, furin, PCSK1 peptidase, PCSK2 peptidase, PCSK4 peptidase, PCSK6 peptidase, PCSK5 peptidase, PCSK7 peptidase, tripeptidyl-peptidase II, cucumisin, prolyl oligopeptidase, dipeptidyl-peptidase IV (eukaryote), acylaminoacyl-peptidase, fibroblast activation protein alpha subunit, oligopeptidase B, carboxypeptidase Y, serine carboxypeptidase A, serine carboxypeptidase C, serine carboxypeptidase D, kex carboxypeptidase, D-Ala-D-Ala carboxypeptidase A, K15-type DD-transpeptidase, D-Ala-D-Ala carboxypeptidase B, aminopeptidase DmpB, D-Ala-D-Ala peptidase C, peptidase C1p (type 1), Xaa-Pro dipeptidyl-peptidase, Lon-A peptidase, PIM1 peptidase, assemblin, cytomegalovirus assemblin, herpesvirus 8-type assemblin, repressor LexA, UmuD protein, signal peptidase I, mitochondrial inner membrane peptidase 1, signal peptidase SipS, signalase (subject) 21 kDa component, lysosomal Pro-Xaa carboxypeptidase, dipeptidyl-peptidase II, hepacivirin, potyvirus P1 peptidase, pestivirus NS3 polyprotein peptidase, equine arteritis virus serine peptidase, prolyl aminopeptidase, C-terminal processing peptidase-1, C-terminal processing peptidase-2, tricorn core peptidase (archaea), signal peptide peptidase A, infectious pancreatic necrosis bimavirus Vp4 peptidase, dipeptidase E, sedolisin, sedolisin-B, tripeptidyl-peptidase I, kumamolisin, physarolisin, SpoIVB peptidase, archaean proteasome, beta component, bacterial proteasome, beta component, HslV component of HslUV peptidase, constitutive proteasome catalytic subunit 1, constitutive proteasome catalytic subunit 2, constitutive proteasome catalytic subunit 3, gamma-glutamyltransferase 1 (bacterial-type), murein tetrapeptidase LD-carboxypeptidase (Escherichia-type), PepA aminopeptidase, presenilin 1, polyporopepsin, canditropsin, candidapepsin SAP2, caspase-2, caspase DRONC (Drosophila melanogaster)-type peptidase, ubiquitin-specific peptidase 7, human coronavirus 229E main peptidase, SARS coronavirus picornain 3C-like peptidase, AvrPphB peptidase, sortase B, psychrophilic alkaline metallopeptidase (Pseudomonas sp.), acutolysin A, aminopeptidase S (Staphylococcus-type), carboxypeptidase Pfu, isoaspartyl dipeptidase (metallo-type), D-aminopeptidase DppA, and murein endopeptidase. In certain embodiments, the enzyme cleavable moiety is cleavable by a cathepsin protease or peptidase.

### Compositions and Methods for Formulating Pharmaceutical Compositions

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more compounds or a salt thereof. In certain embodiments, a pharmaceutical composition comprises a compound described herein and a pharmaceutically acceptable diluent or carrier. In certain embodiments, a pharmaceutical composition comprises a sterile saline solution and one or more compound described herein. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound described herein and sterile water. In certain embodiments, a pharmaceutical composition consists of one compound described herein and sterile water. In certain embodiments, the sterile water is pharmaceutical grade water. In certain embodiments, a pharmaceutical composition comprises one or more compound described herein and phosphate-buffered saline (PBS). In certain embodiments, a pharmaceutical composition consists of one or more compound described herein and sterile PBS. In certain embodiments, the sterile PBS is pharmaceutical grade PBS.

Pharmaceutical compositions comprising compounds described herein encompass any pharmaceutically acceptable salts, esters, or salts of such esters, or any other oligonucleotide which, upon administration to a subject, including a human, is capable of providing (directly or indirectly) the biologically active metabolite or residue thereof. Certain embodiments are drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Non-Limiting Disclosure and Incorporation by Reference

While certain compounds, compositions and methods described herein have been described with specificity in accordance with certain embodiments, the following examples serve only to illustrate the compounds described herein and are not intended to limit the same.

Each reference recited herein, including but not limited to scientific literature, patent publications, GenBank accession numbers, and the like is incorporated by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “ATmCGAUCG,” wherein mC indicates a cytosine base comprising a methyl group at the 5-position.

Compounds described herein include (R) or (S), as α or β such as for sugar anomers, or as (D) or (L) such as for amino acids etc. Included in the compounds provided herein are all such possible isomers, including their racemic and optically pure forms, unless specified otherwise. Likewise, all cis- and trans-isomers and tautomeric forms are also included. Compounds described herein include chirally pure or enriched mixtures as well as racemic mixtures. For example, oligonucleotides having a plurality of phosphorothioate internucleoside linkages include such compounds in which chirality of the phosphorothioate internucleoside linkages is controlled or is random.

Unless otherwise indicated, any compound, including oligomeric compounds, described herein includes a pharmaceutically acceptable salt thereof.

Compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S.

### EXAMPLES

### Example 1: Design of a Modified Oligonucleotide Conjugated to Angiotensin II Peptides

Compound Nos. 1229761 and 1229762 were designed with the N-terminus of an Angiotensin II peptide ([N6-(2-azidoacetyl)-K]DRVYIHPF (SEQ ID NO: 14) and [N6-(2-azidoacetyl)-K]PPPAGSSPGDRVYIHPF (SEQ ID NO: 15), respectively) conjugated to a 5′-end of a modified oligonucleotide via a linker as represented in Table 1 below. The modified oligonucleotide has a sugar motif as shown in Table 1. Letters indicate nucleobase sugar modifications as follows: ‘e’ represents a 2′-MOE sugar moiety, ‘d’ represents a 2′-β-D-deoxyribosyl sugar moiety, and ‘k’ represents a cET sugar moiety. The internucleoside linkages of the modified oligonucleotide below are denoted by ‘p’, which represents a phosphodiester internucleoside linkage or ‘s’, which represents a phosphorothioate internucleoside linkage. All cytosine residues are 5-methylcytosines.

Compound Nos. 1229761 and 1229762 described in Table 1 each consists of a 19-nucleotide sequence (from 5′ to 3′): TCAGCATTCTAATAGCAGC (SEQ ID NO: 8), wherein nucleosides 4 to 19 comprise the sequence GCATTCTAATAGCAGC (SEQ ID NO: 9) which is 100% complementary to mouse MALAT1 RNA (GENBANK Accession No. NT_082868.4 truncated from nucleotides 2689000 to 2699000 (SEQ ID NO: 1)). Nucleosides 1 to 3 of Compound Nos. 1229761 and 1229762 described in Table 1 comprise of the sequence TCA to form a nuclease cleavable linker. The 5′-most nucleoside “T” is conjugated to the N-terminus of the Angiotensin II peptide. The attachment point of the Angiotensin II peptide to the linker is an underlined “K” (which represents an N6-(2-azidoacetyl)-lysine) in the table below. Compound No. 1295887 has the same nucleobase sequence, sugar motif, internucleoside linkage pattern, and Angiotensin II peptide as Compound No. 1229762, and differs only in that 1295887 contains a 3′ Cy3 label.

Control Compound No. 556089 is 100% complementary to mouse MALAT RNA (GENBANK Accession No. NT_082868.4 truncated from nucleotides 2689000 to 2699000 (SEQ ID NO: 1)) at positions 6554 to 6569. The sugar moieties of Compound No. 556089 are represented by ‘d’, which represents a 2′-D-deoxyribosyl sugar moiety, or ‘k’, which represents a cET sugar moiety. The internucleoside linkages of the modified oligonucleotide below are denoted ‘p’, which represents a phosphodiester internucleoside linkage or ‘s’, which represents a phosphorothioate internucleoside linkage. All cytosine residues are 5-methylcytosines.

Control Compound No. 1213287 described in Table 1 consists of a 19-nucleotide sequence (from 5′ to 3′): TCAGCATTCTAATAGCAGC (SEQ ID NO: 8), wherein nucleosides 4 to 19 comprise the sequence GCATTCTAATAGCAGC (SEQ ID NO: 9) which is 100% complementary to mouse MALAT1 RNA (GENBANK Accession No. NT_082868.4 truncated from nucleotides 2689000 to 2699000 (SEQ ID NO: 1)). Compound No. 1213287 contains a 3′ Cy3 label.

Compound No. 556089 has the same nucleobase sequence, sugar motif, and internucleoside linkage pattern as nucleosides 4 to 19 of Compound Nos. 1229761, 1229762, and 1295887. Compound Nos. 1229761, 1229762, and 1295887 differ from Compound No. 556089 in that Compound Nos. 1229761 and 1229762 have three 2′-β-D-deoxyribosyl sugar moieties linking it to an Angiotensin II peptide.

TABLE 1
Design of a modified oligonucleotide conjugated to Angiotensin II peptides

| | | | Internucleoside | | | | | |
|---|---|---|---|---|---|---|---|---|
| | | SEQ | linkage | Sugar | | SEQ | | |
| Compound | Sequence | ID | backbone | (5′ to | 5’- | ID | | |
| No. | (5′ to 3′) | NO | (5’ to 3′) | 3′) | conjugate | NO | 5′-linker | 3’-label |
| 1229761 | TCAGCAT | 8 | ppps | dddk | KDRV | 14 | (p)-6- | None |
| | TCTAATA | | ssss | kkdd | YIHP | | aminohexanol- | |
| | GCAGC | | ssss | dddd | F | | 1- | |
| | | | ssss | dddd | | | carboxymethyl | |
| | | | ss | kkk | | | [triazolo | |
| | | | | | | | BCN1] | |
| | | | | | | | carbamate | |
| 1229762 | TCAGCAT | 8 | ppps | dddk | KPPP | 15 | (p)-6- | None |
| | TCTAATA | | ssss | kkdd | AGSS | | aminohexanol- | |
| | GCAGC | | ssss | dddd | PGDR | | 1- | |
| | | | ssss | dddd | VYIH | | carboxymethyl | |
| | | | ss | kkk | PF | | [triazolo | |
| | | | | | | | BCN1] | |
| | | | | | | | carbamate | |
| 1295887 | TCAGCAT | 8 | ppps | dddk | KPPP | 15 | (p)-6- | Cy3 |
| | TCTAATA | | ssss | kkdd | AGSS | | aminohexanol- | |
| | GCAGC | | ssss | dddd | PGDR | | 1- | |
| | | | ssss | dddd | VYIH | | carboxymethyl | |
| | | | ss | kkk | PF | | [triazolo | |
| | | | | | | | BCN1] | |
| | | | | | | | carbamate | |
| 556089 | GCATTCT | 9 | ssss | kkkd | None | | None | None |
| | AATAGCA | | ssss | dddd | | | | |
| | GC | | ssss | dddd | | | | |
| | | | sss | dkkk | | | | |
| 1213287 | TCAGCAT | 8 | ppps | dddk | None | | None | Cy3 |
| | TCTAATA | | ssss | kkdd | | | | |
| | GCAGC | | ssss | dddd | | | | |
| | | | ssss | dddd | | | | |
| | | | ss | kkk | | | | |

### Example 2: Activation of AGTR1 Signaling by Angiotensin II Conjugated Modified Oligonucleotides

Activated AGTR1 couples to the G_{αq} subunit of heterotrimeric G proteins, which activates the phospholipase C (PLC) and subsequently inositol 1,4,5-trisphosphate (IP3) signaling cascade. AGTR1 signaling was measured using an IP-One Gq kit (cisbio #62IPAPEB). The kit detects the accumulation of inositol monophosphate (IP1), a stable downstream metabolite of IP3 induced by activation of a phospholipase C (PLC) cascade.

Compounds tested include Compound Nos. 556089, 1229761, and 1229762 described herein above. An additional control compound, AngII, which consists of the peptide motif DRVYIHPF, was also tested. HEK293 cells expressing FAP tagged human AGTR1 (HEK-AGTR1 FAP cells from Spectragenetics) were cultured with AngII, Compound No. 556089, Compound No. 1229761, and Compound No. 1229762 at the concentrations detailed in Table 2 for 90 minutes. AGTR1 activation was measured using the IP-One Gq kit. Compound Nos. 1229761 and 1229762 both show activation of the receptor in comparison to the unconjugated parent compound 556089. The extended angiotensinogen peptide conjugated modified oligonucleotide (1229762) showed enhanced activation compared to 1229761.

As a negative control, Compound Nos. 556089 and 1229761 were also tested in HEK293 cells expressing FAP tagged human Apelin receptor (HEK-APLNR FAP cells from Spectragenetics). The HEK-APLNR FAP do not express AGTR1. HEK-APLNR FAP cells were cultured with Compound Nos. 556089 and 1229761, described herein above, at the concentrations detailed in Table 2 for 24 hours. As seen in Table 2, AGTR1 activation was not observed in the HEK-APLNR FAP cells with any of the compounds tested.

TABLE 2
Activation of AGTR1

| | IP1 accumulation (nM) | | | | | | |
|---|---|---|---|---|---|---|---|
| Concentration | HEK-AGTR1 FAP cells | | | | HEK-APLNR FAP cells | | |
| (nM) | AngII | 556089 | 1229761 | 1229762 | 556089 | 1229761 | 1229762 |
| 0.0256 | 1183 | 696 | 647 | 721 | 584 | 590 | 723 |
| 0.128 | 2569 | 669 | 688 | 967 | 536 | 477 | 634 |
| 0.64 | 1710 | 692 | 775 | 1292 | 563 | 564 | 679 |
| 3.2 | 4465 | 658 | 1248 | 2514 | 538 | 510 | 596 |
| 16 | 8913 | 646 | 3101 | 5558 | 526 | 505 | 526 |
| 80 | 15383 | 605 | 8151 | 13794 | 514 | 492 | 517 |
| 400 | 13890 | 648 | 11368 | 16811 | 553 | 425 | 609 |
| 2000 | 14825 | 672 | 13100 | 17550 | 542 | 472 | 576 |

### Example 3: Activity of an Angiotensin II Conjugated Modified Oligonucleotide on MALAT RNA in Vitro, Multiple Doses

HEK293 cells expressing FAP tagged human AGTR1 (HEK-AGTR1 FAP cells from Spectragenetics) were treated with Compound Nos. 556089, 1229761, and 1229762, described herein above, by free uptake at the concentrations detailed in Table 3 for 24 hours. At the end of the treatment period, total RNA was isolated from the cells and MALAT1 RNA levels were measured by quantitative real-time PC&. Human MALAT1 primer probe set RTS2737 (forward sequence AAGAGGCGGCGGAAGGT (SEQ ID NO: 2); reverse sequence CGGGCGAGGCGTATITATAG (SEQ ID NO: 3); probe sequence TCCGGTGATGCGAGTITGTITCTCCG (SEQ ID NO: 4)) was used to measure RNA levels. MALAT1 RNA levels were normalized according to total RNA content, as measured by RIBOGREEN®. Reduction of MALAT1 RNA is presented Table 3 below as percent MALAT1 RNA amount relative to untreated control (UTC) cells. The half maximal inhibitory concentration (IC₅₀) of each modified oligonucleotide was calculated using a non-linear fit [inhibitor] vs. response—variable slope (four parameters) formula in GraphPad Prism 7.01.

The data in Table 3 shows that Compound Nos. 1229761 and 1229762, the modified oligonucleotides conjugated to the angiotensin II peptides, shows significantly greater reduction of target RNA compared to the parent unconjugated oligonucleotide, Compound No. 556089. Compound No. 1229762, with the extended N-terminal angiotensinogen peptide conjugate shows the greatest improvement in target knockdown.

TABLE 3
Dose-dependent percent reduction of human
MALAT1 RNA by modified oligonucleotides

| Concentration | MALAT (% control) | | | IC50 (nM) | | |
|---|---|---|---|---|---|---|
| (nM) | 556089 | 1229761 | 1229762 | 556089 | 1229761 | 1229762 |
| 0 | 100 | 100 | 100 | 850 | 182 | 100 |
| 12 | 86 | 126 | 105 | | | |
| 37 | 98 | 125 | 90 | | | |
| 111 | 91 | 76 | 59 | | | |
| 333 | 63 | 63 | 36 | | | |
| 1000 | 60 | 67 | 41 | | | |
| 3000 | 36 | 36 | 22 | | | |
| 9000 | 29 | 33 | 24 | | | |

### Example 4: Activity of an Angiotensin II Conjugated Modified Oligonucleotide on MALAT RNA in Vitro, Multiple Doses

HEK293 cells expressing FAP tagged human AGTR1 (HEK-AGTR1 FAP cells from Spectragenetics) were treated with Compound Nos. 556089, 1229761, and 1229762, described herein above, by free uptake at the concentrations detailed in Table 4 for 48 hours. At the end of the treatment period, total RNA was isolated from the cells and MALAT1 RNA levels were measured by quantitative real-time PCR. Human MALAT1 primer probe set RTS2737 (forward sequence AAGAGGCGGCGGAAGGT (SEQ ID NO: 2); reverse sequence CGGGCGAGGCGTATITATAG (SEQ ID NO: 3); probe sequence TCCGGTGATGCGAGTTGTTCTCCG (SEQ ID NO: 4)) was used to measure RNA levels. MALAT1 RNA levels were normalized according to total RNA content, as measured by RIBOGREEN®. Reduction of MALAT1 RNA is presented Table 4 below as percent MALAT1 RNA amount relative to untreated control (UTC) cells. The half maximal inhibitory concentration (IC₅₀) of each modified oligonucleotide was calculated using a non-linear fit [inhibitor] vs. response-variable slope (three parameters) formula in GraphPad Prism 7.01.

The data in Table 4 show that Compound Nos. 1229761 and 1229762, the modified oligonucleotide conjugated to the angiotensinogen peptides, shows significantly greater reduction of target RNA compared to the parent unconjugated oligonucleotide, Compound No. 556089.

TABLE 4
Dose-dependent percent reduction of human
MALAT1 RNA by modified oligonucleotides

| Concentration | MALAT (% control) | | | IC50 (nM) | | |
|---|---|---|---|---|---|---|
| (nM) | 556089 | 1229761 | 1229762 | 556089 | 1229761 | 1229762 |
| .10 | 104 | 85 | 103 | 1530 | 290 | 125 |
| .3 | 106 | 99 | 106 | | | |
| .9 | 101 | 91 | 101 | | | |
| 2.7 | 97 | 94 | 100 | | | |
| 8.2 | 100 | 98 | 80 | | | |
| 24.7 | 104 | 92 | 69 | | | |
| 74.1 | 99 | 85 | 66 | | | |
| 222.2 | 82 | 53 | 42 | | | |
| 666.7 | 68 | 26 | 16 | | | |
| 2000 | 43 | 18 | 12 | | | |
| 6000 | 25 | 16 | 9 | | | |

### Example 5: Activity of an Angiotensin II Conjugated Modified Oligonucleotide on MALAT RNA In Vivo, Multiple Dose

Compound Nos. 556089, 1229761, and 1229762 are described in Table 1 above. Compound Nos. 556089, 1229761, and 1229762 were tested in C57BL6/J mice. The mice were divided into groups of 4 mice each. Each mouse in each group received a tail vein IV delivery of either Compound No. 556089, Compound No. 1229761, or Compound No. 1229762 at 0.4, 1.2, or 3.6 μmol/kg at day 0 (first dose), day 4, and day 8. The mice were then sacrificed on day 12. Heart, adipose, adrenal, kidney, and liver tissue was then collected and homogenized and RNA analysis was performed. A group of 4 mice received PBS as a negative control.

### RNA Analysis

RNA was extracted from heart, adipose, adrenal, kidney, and liver tissue for real-time PCR analysis of measurement of RNA expression of MALAT using primer probe set mMALAT1 #2 (forward sequence TGGGTITAGAGAAGGCGTGTACTG (SEQ ID NO: 5); reverse sequence TCAGCGGCAACTGGGAAA (SEQ ID NO: 6); probe sequence CGTTGGCACGACACCTITCAGGGACT (SEQ ID NO: 7)). Results are presented as percent change of RNA, relative to PBS control, normalized to mouse GAPDH.

As shown in Tables 5A and 5B below, treatment with peptide-conjugated modified oligonucleotide resulted enhanced knockdown of MALAT1 in comparison to the parent compound in multiple tissues.

TABLE 5A
Dose-dependent percent reduction of mouse MALAT RNA in vivo

| | | Heart | | Adipose | | Adrenal | |
|---|---|---|---|---|---|---|---|
| | | MALAT-1 | | MALAT-1 | | MALAT-1 | |
| Compound | Dose | RNA | ED₅₀ | RNA | ED₅₀ | RNA | ED₅₀ |
| No. | (μmol/kg) | (% control) | (μmol/kg) | (% control) | (μmol/kg) | (% control) | (μmol/kg) |
| 556089 | 0.4 | 76.7 | 4.11 | 68.4 | 0.034 | 95.2 | 1.102 |
| | 1.2 | 74.7 | | 41.1 | | 75.7 | |
| | 3.6 | 45.7 | | 27.3 | | 64.1 | |
| 1229761 | 0.4 | 82.3 | 2.686 | 66.2 | 0.1 | 90.3 | 1.091 |
| | 1.2 | 68.7 | | 33.0 | | 59.7 | |
| | 3.6 | 31.7 | | 15.5 | | 41.5 | |
| 1229762 | 0.4 | 86.2 | 3.14 | 58.3 | 1.385 | 87.8 | 1.2 |
| | 1.2 | 73.4 | | 40.1 | | 66.5 | |
| | 3.6 | 40 | | 20.5 | | 45.2 | |

TABLE 5B
Dose-dependent percent reduction of mouse MALAT RNA in vivo

| | | Kidney | | Liver | |
|---|---|---|---|---|---|
| | Dose | MALAT-1 | ED₅₀ | MALAT-1 | ED₅₀ |
| Compound | (μmol/ | RNA | (μmol/ | RNA | (μmol/ |
| No. | kg) | (% control) | kg) | (% control) | kg) |
| 556089 | 0.4 | 50.0 | 3.535 | 35.8 | 1.578 |
| | 1.2 | 46.7 | | 24.1 | |
| | 3.6 | 24.6 | | 7.2 | |
| 1229761 | 0.4 | 53.8 | 1.88 | 34.7 | 0.063 |
| | 1.2 | 39.6 | | 12 | |
| | 3.6 | 21.4 | | 5.7 | |
| 1229762 | 0.4 | 50.6 | 3.084 | 40.5 | 0.085 |
| | 1.2 | 43.8 | | 15.1 | |
| | 3.6 | 22.4 | | 5.7 | |

### Example 6: Activity of an Angiotensin II Conjugated Modified Oligonucleotide on MALAT RNA In Vivo in the Presence of an AGTR1 Antagonist

Compound Nos. 1213287 and 1295887 are described in Table 1 above. Compound Nos. 1213287 and 1295887 were tested in C57BL6/J mice with or without pretreatment with the AGTR1 antagonist, Losartan. The mice were divided into groups of 3 mice each. Mice receiving Losartan were treated with 150 mg/L in the drinking water for 10 days. Each mouse received a single IV dose on day 10 of either Compound No. 1213287 or 1295887 of either 1.2 or 3.6 μmol/kg. The mice were then sacrificed on day 14. Heart, adipose, adrenal, kidney, and liver tissue was then collected and homogenized and RNA analysis was performed. A group of 4 mice received PBS as a negative control.

### RNA Analysis

RNA was extracted from heart, adipose, adrenal, kidney, and liver tissue for real-time PCR analysis of measurement of RNA expression of MALAT using primer probe set mMALAT1 #2 (forward sequence TGGGTITAGAGAAGGCGTGTACTG (SEQ ID NO: 5); reverse sequence TCAGCGGCAACTGGGAAA (SEQ ID NO: 6); probe sequence CGTIGGCACGACACCTICAGGGACT (SEQ ID NO: 7)). Results are presented as percent change of RNA, relative to PBS control, normalized to mouse GAPDH.

As shown in Table 6 below, antagonism of AGTR1 with Losartan reduced the activity of the oligonucleotides conjugated to Angiotensin II peptide, which suggested a requirement for AGTR1 for productive uptake of Ang II conjugates.

TABLE 6
Reduction of AngII conjugate activity with AGTR1 blockade

| Compound | Dose | MALAT RNA (% control) | | | | |
|---|---|---|---|---|---|---|
| No. | (μmol/kg) | Heart | Adipose | Adrenal | Kidney | Liver |
| 1213287 | 1.2 | 77.0 | 78.0 | 78.7 | 58.3 | 53.7 |
| | 3.6 | 57.3 | 53.0 | 50.0 | 42.3 | 35.0 |
| 1213287 + | 1.2 | 80.3 | 79.0 | 67.7 | 52.7 | 49.3 |
| losartan | 3.6 | 61.7 | 50.0 | 63.7 | 42.7 | 40.3 |
| 1295887 | 1.2 | 61.7 | 55.3 | 57.0 | 40.3 | 43.7 |
| | 3.6 | 42.0 | 37.0 | 36.7 | 35.3 | 27.3 |
| 1295887 + | 1.2 | 84.0 | 106.3 | 91.3 | 86.3 | 76.3 |
| losartan | 3.6 | 87.7 | 82.7 | 80.3 | 70.7 | 83.7 |

What is claimed:

1. A compound comprising a modified oligonucleotide linked to an angiotensin II type I receptor (AGTR1) binding conjugate moiety.

2. The compound of claim 1, wherein the modified oligonucleotide is linked to the AGTR1 binding conjugate moiety via a conjugate linker.

3. The compound of claim 1, wherein the AGTR1 binding conjugate moiety comprises a AGTR1 cell-targeting moiety.

4. The compound of claim 1, wherein the AGTR1 binding conjugate moiety consists of a AGTR1 cell-targeting moiety.

5. The compound of claim 1, wherein the AGTR1 binding conjugate moiety comprises an AGTR1 cell-targeting moiety and a peptide extender.

6. A compound comprising,
a) a modified oligonucleotide; and
b) a conjugate group comprising a conjugate moiety and a conjugate linker,
wherein the conjugate moiety comprises an AGTR1 binding cell-targeting moiety and a peptide extender, and
wherein the conjugate linker links the conjugate moiety to the oligonucleotide via the peptide extender.

7. The compound of any of claims 1-6, wherein the AGTR1 binding cell-targeting moiety is a peptide cell-targeting moiety, small molecule cell-targeting moiety, aptamer cell-targeting moiety, or antibody cell-targeting moiety targeted to AGTR1.

8. The compound of any of claims 1-6, wherein the AGTR1 binding cell-targeting moiety comprises a peptide represented by amino acid sequence A₁A₂A₃A₄A₅A₆A₇A₈ (SEQ ID NO: 11), wherein
A₁ is selected from Asp, Sar, Ala, and NH₂;
A₂ is selected from Arg and Gln;
A₃ is selected from Val and Ala;
A₄ is selected from Tyr, Ala, Ile, Gly, Cha;
A₅ is selected from Ile and Val;
A₆ is selected from His and Ala;
A₇ is selected from Pro and Ala; and
A₈ is selected from Phe, Ala, Ile, Gly, Cha, and Dip,
wherein Sar is N-methylglycine, Cha is beta-cyclohexylalanine, and Dip is diphenylalanine.

9. The compound of claim 8, wherein A₁A₂A₃A₄A₅A₆A₇A₈ (SEQ ID NO: 11) is Asp-Arg-Val-Tyr-Ile-His-Pro-Phe (SEQ ID NO: 12).

10. The compound of claim 8, wherein A₁A₂A₃A₄A₅A₆A₇A₈ (SEQ ID NO: 11) is Asp-Arg-Val-Tyr-Val-His-Pro-Phe (SEQ ID NO: 13).

11. The compound of any of claims 2-10, wherein the modified oligonucleotide is attached to the cell-targeting moiety or the peptide extender via click chemistry, via a disulfide bridge, or via a maleimide linker.

12. The compound of any of claims 2-11, wherein the conjugate linker is selected from the group consisting of pyrrolidine, 8-amino-3,6-dioxaoctanoic acid (ADO), succinimidyl 4-(N-maleimidomethyl) cyclohexane-1-carboxylate (SMCC), and 6-aminohexanoic acid.

13. The compound of any of claims 2-11, wherein the conjugate linker is (p)-6-aminohexanol-1-carboxymethyl[triazoloBCN1]carbamate.

14. The compound of any of claims 1-13, wherein the AGTR1 binding cell-targeting moiety is a peptide cell-targeting moiety comprising a modified lysine that links the peptide cell-targeting moiety to the conjugate linker.

15. The compound of claim 14, wherein the modified lysine is linked to the amino terminus of A₁.

16. The compound of claim 14, wherein the modified lysine is linked to the carboxy terminus of A₈.

17. The compound of claim 14, wherein the modified lysine comprises an azide.

18. The compound of claim 14, wherein the modified lysine is azido-acetyl-lysine.

19. The compound of any of claims 6-18, wherein the conjugate group comprises the sequence selected from [N6-(2-azidoacetyl)-K]DRVYIHPF (SEQ ID NO: 14), [N6-(2-azidoacetyl)-K]PPPAGSSPGDRVYIHPF (SEQ ID NO: 15), XDRVYIHPF (SEQ ID NO: 16), and XPPPAGSSPGDRVYIHPF (SEQ ID NO: 17), wherein X is selected from lysine, D-lysine, L-lysine, and N6-(2-azidoacetyl)-lysine.

20. The compound of any one of claims 6-18, wherein the peptide extender comprises or consists essentially of 3 to 50, 3 to 45, 3 to 40, 3 to 35, 3 to 30, 3 to 25, 3 to 20, 3 to 15, 3 to 10, 6 to 50, 6 to 45, 6 to 40, 6 to 35, 6 to 30, 6 to 25, 6 to 20, 6 to 15, or 6 to 10 amino acids.

21. The compound of any one of claims 6-18, wherein the peptide extender comprises at least 6, at least 7, at least 8, at least 9, or at least 10 amino acids.

22. The compound of any one of claims 6-18, wherein the peptide extender comprises at least one, at least 2 or at least 3 amino acids selected from serine, proline, hydroxyproline, methionine, cysteine and tyrosine.

23. The compound of claim 22, wherein the at least 2 or at least 3 amino acids are contiguous.

24. The compound of any one of claims 6-18, wherein the peptide extender comprises three contiguous proline residues.

25. The compound of any one of claims 6-18, wherein the peptide extender has a molecular weight of about 400 g/mol to about 1800 g/mol.

26. The compound of any one of claims 2-25, wherein the conjugate linker is connected to the 5′ end of the oligonucleotide.

27. The compound of any one of claims 2-25, wherein the conjugate linker is connected to the 3′ end of the oligonucleotide.

28. The compound of any one of claims 2-25, wherein the conjugate linker is connected to a carboxy terminus of the peptide extender.

29. The compound of any one of claims 2-25, wherein the conjugate linker is connected to an amino terminus of the peptide extender.

30. The compound of any one of claims 5-29, wherein the peptide extender has an amino acid sequence selected from: X₁PPPAGSSPG (SEQ ID NO: 30), X₂PPPAGSSPG (SEQ ID NO: 31), X₁X₂PPAGSSPG (SEQ ID NO: 32), X₁PX₂PAGSSPG (SEQ ID NO: 33), X₁PPX₂AGSSPG (SEQ ID NO: 34), X₁PPPX₂GSSPG (SEQ ID NO: 35), X₁PPPAX₂SSPG (SEQ ID NO: 36), X₁PPPAGX₂SPG (SEQ ID NO: 37), X₁PPPAGSX₂PG (SEQ ID NO: 38), X₁PPPAGSSX₂G (SEQ ID NO: 39), and X₁PPPAGSSP X₂ (SEQ ID NO: 40), wherein X₁ is selected from selected from lysine, D-lysine, L-lysine, and N6-(2-azidoacetyl)-lysine and X₂ is any amino acid.

31. The compound of any one of claims 5-29, wherein the peptide extender has an amino acid sequence that is at least 70%, at least 75%, at least 80%, at least 85%, at least 90%, at least 95%, or 100% identical to the amino acid sequence of X₁PPPAGSSPG (SEQ ID NO: 30), wherein X₁ is selected from selected from lysine, D-lysine, L-lysine, and N6-(2-azidoacetyl)-lysine.

32. The compound of any one of claims 5-29, wherein the peptide extender has an amino acid sequence selected from: CPPPAGSSPG (SEQ ID NO: 41), XPPPAGSSPG (SEQ ID NO: 31), CXPPAGSSPG (SEQ ID NO: 42), CPXPAGSSPG (SEQ ID NO: 43), CPPXAGSSPG (SEQ ID NO: 44), CPPPXGSSPG (SEQ ID NO: 45), CPPPAXSSPG (SEQ ID NO: 46), CPPPAGXSPG (SEQ ID NO: 47), CPPPAGSXPG (SEQ ID NO: 48), CPPPAGSSXG (SEQ ID NO: 49), and CPPPAGSSPX (SEQ ID NO: 50), wherein X is any amino acid.

33. The compound of any one of claims 5-29, wherein the peptide extender comprises an amino acid sequence that is at least 70%, at least 75%, at least 80%, at least 85%, at least 90%, at least 95%, or 100% identical to the amino acid sequence of CPPPAGSSPG (SEQ ID NO: 41).

34. The compound of any one of claims 5-29, wherein the peptide extender is represented by amino acid sequence CAGSIKPPPAGSSPG (SEQ ID NO: 51) or KAGSIKPPPAGSSPG (SEQ ID NO: 52).

35. The compound of any one of claims 5-29, wherein the peptide extender has an amino acid sequence selected from: XPAPSGPSPG (SEQ ID NO: 53), XAGSIKPPPAGSSPG (SEQ ID NO: 54), and XAGMSGASAG (SEQ ID NO: 55), wherein X is selected from lysine, D-lysine, L-lysine, and N6-(2-azidoacetyl)-lysine, and cysteine.

36. The compound of any one of claims 30, 31, or, 34, wherein the lysine of the peptide extender is D-Lysine.

37. The compound of any one of claims 3-31, wherein the peptide extender has a net charge of 0, 1, or 2 at neutral pH.

38. The compound of any one of claims 1-37, wherein the AGTR1 binding conjugate moiety consists or consists essentially of the peptide cell-targeting moiety.

39. The compound of any of claims 1-38, wherein the modified oligonucleotide is 8 to 80 linked nucleosides in length.

40. The compound of any of claims 1-38, wherein the modified oligonucleotide is 10 to 30 linked nucleosides in length.

41. The compound of any of claims 1-38, wherein the modified oligonucleotide is 12 to 30 linked nucleosides in length.

42. The compound of any of claims 1-38, wherein the modified oligonucleotide is 15 to 30 linked nucleosides in length.

43. The compound of any one of claims 1-38, wherein the modified oligonucleotide comprises at least one modified internucleoside linkage, at least one modified sugar, and/or at least one modified nucleobase.

44. The compound of claim 43, wherein the modified internucleoside linkage is a phosphorothioate internucleoside linkage.

45. The compound of claim 44, wherein each internucleoside linkage of the modified oligonucleotide is a phosphorothioate internucleoside linkage.

46. The compound of claim 43, wherein the modified sugar is a bicyclic sugar.

47. The compound of claim 46, wherein the bicyclic sugar is selected from the group consisting of: 4′-(CH2)—O-2′ (LNA); 4′-(CH2)2-O-2′ (ENA); and 4′-CH(CH3)—O-2′ (cEt).

48. The compound of claim 46, wherein the bicyclic sugar is in the β-D configuration.

49. The compound of claim 43, wherein the modified sugar is a non-bicyclic sugar.

50. The compound of claim 49, wherein the non-bicyclic sugar is selected from the group consisting of 2′-O-methoxyethyl, 2′-F, and 2′-OMe.

51. The compound of claim 43, wherein the modified nucleobase is a 5-methylcytosine.

52. The compound of any one of claims 1-51, wherein the modified oligonucleotide comprises:
d) a gap segment consisting of linked deoxynucleosides;
e) a 5′ wing segment consisting of linked nucleosides; and
f) a 3′ wing segment consisting of linked nucleosides;
wherein the gap segment is positioned immediately adjacent to and between the 5′ wing segment and the 3′ wing segment and wherein each nucleoside of each wing segment comprises a modified sugar.

53. The compound of any one of claims 1-52, wherein the modified oligonucleotide is single-stranded.

54. The compound of any one of claims 1-53, wherein the modified oligonucleotide is an antisense oligonucleotide.

55. The compound of claim 1 or claim 6, wherein the modified oligonucleotide is a miRNA antagonist or miRNA mimic.

56. The compound of claim 1 or claim 6, wherein the compound comprises a double-stranded duplex.

57. The compound of claim 56, wherein the double-stranded duplex comprises:
c) a first strand comprising the modified oligonucleotide; and
d) a second strand complementary to the first strand.

58. The compound of claim 57, wherein the first strand comprising the modified oligonucleotide is complementary to a RNA transcript.

59. The compound of claim 57 or 58, wherein the second strand is complementary to a RNA transcript.

60. The compound of claim 55, wherein the compound is a miRNA mimic.

61. The compound of any of claims 1-60, wherein the compound comprises at least one ribonucleotide.

62. The compound of any of claims 1-60, wherein the compound comprises at least one β-D-2′ deoxyribosyl sugar moiety.

63. The compound of any of claims 1-62, wherein the modified oligonucleotide is complementary to a RNA transcript.

64. The compound of claim 63, wherein the RNA transcript is pre-mRNA, mRNA, non-coding RNA, or miRNA.

65. A composition comprising the compound of any one of claims 1-64 and a pharmaceutically acceptable carrier or diluent.

66. A composition consisting or consisting essentially of the compound of any one of claims 1-64 and a pharmaceutically acceptable carrier or diluent.

67. The composition of claim 65 or 66, wherein the pharmaceutically acceptable carrier or diluent is phosphate buffered saline (PBS).

68. The compound of any one of claims 1-64, wherein the compound is in a form of a salt.

69. The compound of claim 68, wherein the salt is a sodium salt.

70. A method of modulating the expression of a nucleic acid target in a cell expressing AGTR1 comprising contacting the cell with the compound or composition of any preceding claim, thereby modulating expression of the nucleic acid target in the cell.

71. The method of claim 70, wherein the cell is located on or within a tissue selected from heart, adipose, adrenal gland, liver, and kidney.

72. The method of claim 70 or 71, comprising administering the compound or composition to a subject.

73. The method of claim 72, wherein the subject has a condition or disease of a tissue selected from heart, adipose, adrenal gland, liver, and kidney.

74. The method of claim 73, wherein the subject is at risk of a condition or disease of a tissue selected from heart, adipose, adrenal gland, liver, and kidney.

75. The method of any of claims 70-74, wherein the compound inhibits expression of the nucleic acid target.
